(19) United States
(12) Patent Application Publication
(10) Pub. No.: US 2021/0153927 A1
(43) Pub. Date: May 27, 2021

(54) ELECTROSURGICAL INSTRUMENT WITH COMPLIANT ELASTOMERIC ELECTRODE

(71) Applicant: Intuitive Surgical Operations, Inc., Sunnyvale, CA (US)

(72) Inventors: Adam Ross, Prospect, CT (US); Rolf Weiler, II, Tuebingen (DE); Volker Mayer, Tuebingen (DE)

(21) Appl. No.: 16/625,709

(22) PCT Filed: Jun. 28, 2018
(86) PCT No.: PCT/US18/39912

§ 371 Date: Dec. 21, 2019

Related U.S. Application Data
(60) Provisional application No. 62/527,289, filed on Jun. 30, 2017.

(51) Int. Cl. A61B 18/14; A61B 34/35; A61B 18/12

(52) U.S. Cl. CPC A61B 18/1445; A61B 2018/0063; A61B 18/1206; A61B 34/35

(57) ABSTRACT

A surgical tool for performing telesurgical surgical operations such as cutting, shearing, grasping, engaging, or contacting tissue. The surgical tool comprises a pair of jaws cooperatively rotating open and close about an axis of rotation. The jaws further comprise one or more compliant electrodes electrically communicating with a conductor to deliver electrical energy to tissue engaged by the jaws. The electrodes formed of an elastomeric material impregnated with a conductive material or formed atop an elastomeric material allowing a certain amount of flexibility and thereby maintaining more consistent pressure on the tissue when the jaws engage the tissue.

### CROSS REFERENCE TO RELATED APPLICATIONS

This U.S. Non-provisional patent application claims the benefit of Patent Cooperation Treaty (PCT) patent application no. PCT/US2018/039912 entitled, “ELECTRO-SURGICAL INSTRUMENT WITH COMPLIANT ELASTOMERIC ELECTRODE” filed by inventors Adam Ross et al. on Jun. 28, 2018, which in turn claims the benefit of U.S. Provisional Patent Application No. 62/527,289 filed on Jun. 30, 2017, and similarly titled.

### FIELD

The present invention is generally directed to surgical instruments or tools. In particular, the present invention relates to electrosurgical tools with compliant electrode(s) for use in a teleoperated surgical system for minimally invasive surgical operations.

### BACKGROUND

Minimally invasive surgical techniques generally reduce the amount of extraneous tissue damage during surgical procedures, thereby reducing patient recovery time, discomfort, and deleterious side effects. One effect of minimally invasive surgery, for example, is reduced post-operative hospital recovery times. Because the average hospital stay for a standard surgery is typically significantly longer than the average stay for an analogous minimally invasive surgery, increased use of minimally invasive techniques could save millions of dollars in hospital costs each year. Patient recovery times, patient discomfort, surgical side effects, and time away from work can also be reduced by increasing the use of minimally invasive surgery.

Traditional forms of minimally invasive surgery typically include endoscopy, which is visual examination of a hollow space with a viewing instrument called an endoscope. One of the more common forms of endoscopy is laparoscopy, which is visual examination and/or treatment of the abdominal cavity. In traditional laparoscopic surgery a patient's abdominal cavity is insufflated with gas and cannula sleeves are passed through small incisions in the musculature of the patient's abdomen to provide entry ports through which laparoscopic surgical instruments can be passed in a sealed fashion. Such incisions are typically about ½ inch (about 12 mm) in length.

The laparoscopic surgical instruments generally include a laparoscope for viewing the surgical field and working tools defining end effectors. Typical surgical end effectors include clamps, graspers, scissors, staplers, and needle holders, for example. The working tools are similar to those used in conventional (open) surgery, except that the working end or end effector of each tool is separated from its handle by a long extension tube, typically of about 12 inches (about 300 mm) in length, for example, so as to permit the surgeon to introduce the end effector to the surgical site and to control movement of the end effector relative to the surgical site from outside a patient's body.

To perform a surgical procedure, a surgeon typically passes the working tools or instruments through the cannula sleeves to the internal surgical site and manipulates the instruments from outside the abdomen by sliding them in and out through the cannula sleeves, rotating them in the cannula sleeves, levering (i.e., pivoting) the instruments against the abdominal wall, and actuating the end effectors on distal ends of the instruments from outside the abdominal cavity. The instruments normally pivot around centers defined by the incisions which extend through the muscles of the abdominal wall. The surgeon typically monitors the procedure by means of a television monitor which displays an image of the surgical site captured by the laparoscopic camera. Typically, the laparoscopic camera is also introduced through the abdominal wall so as to capture the image of the surgical site. Similar endoscopic techniques are employed in, for example, arthroscopy, retroperitoneoscopy, pelviscopy, nephroscopy, cystoscopy, cisternoscopy, sinoscopy, hysteroscopy, urethroscopy, and the like.

Although traditional minimally invasive surgical instruments and techniques like those just described have proven highly effective, newer systems may provide even further advantages. For example, traditional minimally invasive surgical instruments often deny the surgeon the flexibility of tool placement found in open surgery. Difficulty is experienced in approaching the surgical site with the instruments through the small incisions. Additionally, the added length of typical endoscopic instruments often reduces the surgeon's ability to feel forces exerted by tissues and organs on the end effector. Furthermore, coordination of the movement of the end effector of the instrument as viewed in the image on the television monitor with actual end effector movement is particularly difficult, since the movement as perceived in the image normally does not correspond intuitively with the actual end effector movement. Accordingly, lack of intuitive response to surgical instrument movement input is often experienced. Such a lack of intuitiveness, dexterity, and sensitivity of endoscopic tools has been found to be an impediment in the increased use of minimally invasive surgery.

Minimally invasive robotic (or “teleoperated”) surgical systems have been developed to increase surgical dexterity as well as to permit a surgeon to operate on a patient in an intuitive manner. Teleoperated surgery is a general term for surgical operations using systems where the surgeon uses some form of remote control, e.g., a servomechanism, or the like, to manipulate surgical instrument movements, rather than directly holding and moving the tools by hand. In such a teleoperated surgical system, the surgeon is typically provided with an image of the surgical site on a visual display at a location remote from the patient. The surgeon can typically perform the surgical procedure at the location remote from the patient while viewing the end effector movement on the visual display during the surgical procedure. While typically viewing a three-dimensional image of the surgical site on the visual display, the surgeon performs the surgical procedures on the patient by manipulating master control devices at the remote location, which master control devices control motion of the remotely controlled instruments.

Typically, such a teleoperated surgical system can be provided with at least two master control devices (one for each of the surgeon's hands), which are normally operatively associated with two robotic arms on each of which a surgical instrument is mounted. Operative communication between master control devices and associated teleoperated surgical arm and instrument assemblies is typically achieved through a control system. The control system typically includes at least one processor which relays input commands from the master control devices to the associated teleoperated arm and instrument assemblies and from the arm and instrument assemblies to the associated master control devices in the case of, e.g., force feedback, or the like.

Teleoperated surgical systems may perform a wide variety of surgical procedures using different surgical tools. For example, to perform electrosurgery, electrosurgical tools may be coupled to the teleoperated arms of the teleoperated surgical system. Electrosurgery refers broadly to a class of medical procedures which rely on the application of high frequency electrical energy to patient tissue to achieve a number of possible effects, such as cutting, coagulation, desiccation, and the like. A typical electrosurgical instrument is capable of treating tissue of an organism with the use of heat produced by electrical energy passing through tissue.

Electrosurgical tools include monopolar electrosurgical tools, bipolar electrosurgical tools, harmonic tools, laser tools, ultrasound tools. Electrosurgical tools, used in teleoperated surgery, are mechanically coupled to a teleoperated arm to control its movement; they are also coupled to an electrosurgical generator so that energy may be applied to tissue at or near its end effectors. For example, in some minimally invasive and teleoperated surgical procedures, tissue in the patient's body must be cauterized and severed. To perform such a procedure, bipolar or monopolar cauterizing grips can be introduced through a trocar to engage the target tissue. Electrical energy, such as radio frequency energy, is delivered to the grips to cauterize the engaged tissue.

Electrical energy delivery may be carried out before, during, and/or after tissue shearing. The delivered electrical energy produces heat capable of treating the tissue. For example, the heat may cauterize the tissue or coagulate blood so as to minimize bleeding during a treatment procedure. Electrosurgical tools may use high frequency alternating currents (AC) such as radio frequency (RF) energy to provide the heat necessary for cauterization and coagulation. High frequency RF energy is preferred to minimize muscular contractions and electrocution. Monopolar devices are typically used in conjunction with a grounding pad wherein one pole of an electrosurgical generator is mounted to the instrument and the other pole is mounted to the grounding pad. The electrical current in monopolar devices travels from the instrument through the patient's body to the grounding pad. Bipolar instruments are typically connected to both poles of the electrosurgical generator. Current flow in bipolar devices is typically limited to tissue near the working end of the bipolar instrument, thereby reducing the risk of damaging non-target tissue.

### BRIEF SUMMARY OF THE INVENTION

The embodiments of the invention are summarized by the claims that follow below.

### BRIEF DESCRIPTION OF THE DRAWINGS

FIG. 1A is a block diagram of a first teleoperated surgical system to perform minimally invasive teleoperated surgical procedures using an electrosurgical tool.

FIG. 1B is a block diagram of a second teleoperated surgical system to perform minimally invasive teleoperated surgical procedures using an electrosurgical tool.

FIG. 1C a perspective view of the teleoperated patient-side system of FIG. 1A

FIG. 2A is a perspective view of a teleoperated surgical manipulator with a plurality of teleoperated surgical arms at least one of which includes a electrosurgical tool.

FIG. 2B illustrates mounting of the electrosurgical tool to an adapter of the teleoperated surgical arm.

FIG. 2C illustrates a top view of the adapter of the teleoperated surgical arm of FIG. 2C to which the electrosurgical tool may be mounted.

FIG. 2D illustrates a back side of an exemplary electrosurgical instrument or tool that interfaces to a teleoperated surgical arm.

FIG. 3A is a perspective view of a teleoperated surgical master control console (surgeons console).

FIG. 3B is a perspective view of an exemplary gimbaled control input wrist pivotally supporting a master grip control handle (also referred to as a master grip control input) for the teleoperated surgical master control console of FIG. 3A to control surgical tools including an electrosurgical tool.

FIG. 3C is a cross-sectional view schematically illustrating the master grip control handle (also referred to as a master grip control input) pivotally coupled to the control input wrist of FIG. 3B.

FIG. 4A is a perspective view of the distal end of an exemplary surgical tool; illustrating an electrosurgical end effector with a cutting tip.

FIG. 4B is a perspective view of opposite side of the electrosurgical end effector shown in FIG. 4A.

FIG. 4C illustrates the electrical surgical end effector of FIG. 4A in an open configuration with a view of the cut electrode.

FIG. 4D illustrates the electrical surgical end effector of FIG. 4B in an open configuration with a view of an elastomeric strip between a sealing electrode.

FIG. 4E is a perspective view of an isolated jaw of an exemplary electrosurgical end effector with a cut electrode and cutting tip.

FIG. 4F is a cross section view of the isolated jaw of FIG. 4E.

FIG. 4G is a perspective view of an isolated jaw of an exemplary electrosurgical end effector with a seal electrode and an elastomeric strip.

FIG. 4H is a cross section view of the isolated jaw of FIG. 4G.

FIG. 4I is a cross section view of an exemplary electrical surgical end effector in an open position.

FIG. 4J is a cross section view of an exemplary electrical surgical end effector in a closed position.

FIG. 4K is a perspective view of an isolated jaw of an exemplary electrosurgical end effector with an elastomeric seal electrode.

FIG. 5A-5C are fontal views of an exemplary electrical surgical end effector with tissue being cut and sealed between the jaws of the end effector.

FIG. 6 is an electrical schematic of an exemplary electrosurgical system.

FIG. 7 is an electrical diagram of an exemplary electrosurgical end effector with three poles for simultaneous application of cutting and sealing energy.

### DETAILED DESCRIPTION OF THE INVENTION

In the following detailed description of the embodiments of the invention, numerous specific details are set forth in order to provide a thorough understanding of the embodiments of the invention. However, it will be obvious to one skilled in the art that the embodiments of the invention may be practiced without these specific details. In other instances well known methods, procedures, components, and circuits have not been described in detail so as not to unnecessarily obscure aspects of the embodiments of the invention.

### INTRODUCTION

Teleoperated surgery may be used to perform a wide variety of surgical procedures, including but not limited to open surgery, neurosurgical procedures (such as stereotaxy), endoscopic procedures (such as laparoscopy, arthroscopy, thoracoscopy), and the like. During these teleoperated surgical procedures, surgeons may use high voltage, low current electrical energy of various wave forms to perform such tasks as cautery, cutting tissue, or sealing a vessel. Electrical energy supply devices (also referred to as electrosurgical generators) are coupled to surgical instruments and are typically activated by a foot pedal switch of a foot pedal. One or more foot pedals in a surgeon's console and their corresponding switches may be used to activate these electrical energy supply devices.

The invention provides methods, systems, and apparatus for use in teleoperated minimally invasive surgical operations. In particular, electrosurgical cutting/shearing instruments and systems, as well as methods of performing minimally invasive teleoperated surgical procedures with such instruments are provided. The instruments of the present invention are capable of treating tissue with heat produced by electrical energy while cutting, sealing, shearing, grasping, engaging, or contacting treatment tissue. The electrosurgical treatment may further reduce bleeding of tissue by cauterizing tissue and coagulating blood, or achieve various other desired effects on the treatment tissue. By providing electrosurgical cutting/shearing instruments for use with a teleoperated surgical system, the apparatus and methods of the present invention enable the advantages associated with electrosurgical cutting/shearing treatment to be combined with the advantages of a minimally invasive teleoperated surgery.

Of particular interest to the present invention, bipolar electrosurgical procedures rely on electrodes of different polarity in close proximity to each other against or into tissue. For example, electrodes placed on opposing blades of a surgical scissor or opposing jaws of a surgical grasper may be brought into close proximity to deliver electrical energy to the tissue between the blades or jaws.

One embodiment of the invention is an electrosurgical tool for use with a minimally invasive teleoperated surgical system. The electrosurgical tool comprises an elongate shaft having a proximal end and a distal end. An interface or tool base is coupled to the proximal end of the shaft.

An end effector, for performing a surgical operation such as cutting, shearing, grasping, engaging, or contacting tissue, is coupled to a distal end of the shaft. In one embodiment the end effector comprises a pair of jaws cooperatively rotating open and close about an axis of rotation similar to the mechanical action of a pair of pliers or scissors. The jaws further comprise one or more electrodes electrically communicating with a conductor to deliver electrical energy to tissue engaged by the jaws. In between the electrodes and the jaw base is an elastomeric material, e.g. silicone. The electrodes are allowed to float atop the elastomeric layer thereby maintaining more consistent pressure on the tissue when the jaws engage the tissue. This added compliance allows manufacturing tolerances to be loosened, resulting in a less expensive surgical jaw with equivalent or superior sealing performance.

The adjective “compliant” as used herein, e.g. “compliant electrode” is defined as yielding or floating thereby allowing the electrode to move slightly in relation to the jaw base.

The interface base generally includes one or more mechanical transmission members configured to engage one or more drivers of the teleoperated surgical system. The transmission members transmit forces from the teleoperated surgical system to the end effector via one or more actuation elements so as to pivotally move the jaws. The elongate shaft defines an internal longitudinally extending passage, the actuation element being slideably housed within the passage extending internally along the shaft. The actuation or articulation element may comprise an actuator rod coupled to a connector rod which in turn couples each jaw. Alternatively a system of pulleys may actuate the connector rod to open and close the jaws. Actuation of the actuator rod and connector rod in a distal direction relative to the shaft moves the jaws apart from one another and actuation of the actuator rod and connector rod in a proximal direction relative to the shaft moves the jaws together.

Other embodiments of the invention involve methods for performing minimally invasive teleoperated surgical procedures with the electrosurgical instruments described above. One method includes connecting a surgical instrument to a teleoperated surgical system. Connecting the surgical instrument to a teleoperated surgical system further includes releasably mounting the surgical instrument on a teleoperated surgical arm. Passing the surgical instrument, having an elongate shaft at one end of which an end effector is mounted, through an entry port in a patient body, and engaging tissue with the end effector. The tissue being engaged between jaws of the end effector. Delivering electrical energy to the tissue engaged by the jaws.

### Teleoperated Surgical Systems

Teleoperated surgery generally involves the use of a robot manipulator that has multiple teleoperated manipulator arms. One or more of the teleoperated manipulator arms often support a teleoperated surgical tool or instrument which may be an electrosurgical tool or a non-electrosurgical tool. One or more of the teleoperated manipulator arms are often used to support a surgical image capture device such as an endoscope (which may be any of a variety of instruments such as a laparoscope, an arthroscope, a hysteroscope, or the like), or, optionally, some other imaging modality (such as ultrasound, fluoroscopy, magnetic resonance imaging, or the like). Typically, the teleoperated manipulator arms will support at least two teleoperated surgical tools corresponding to the two hands of a surgeon and one image capture device.

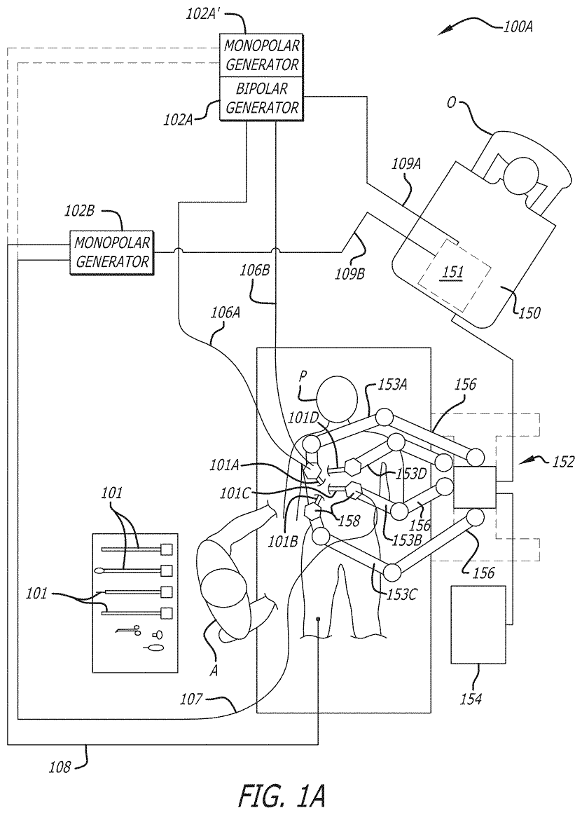

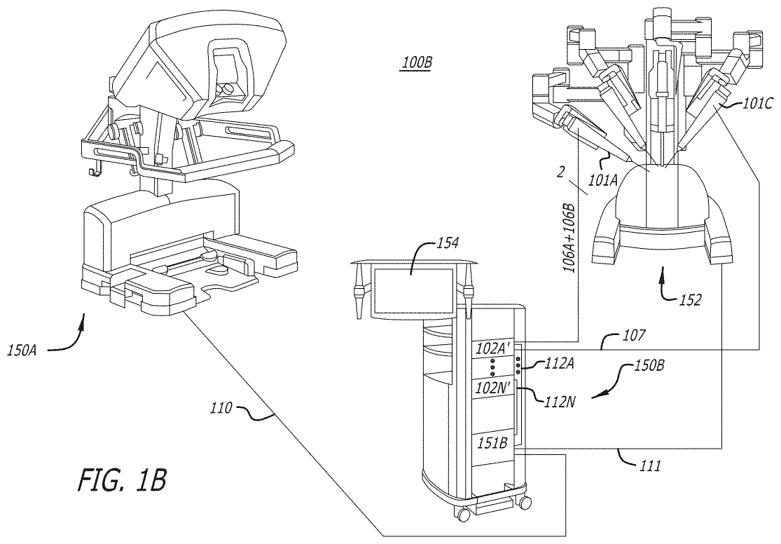

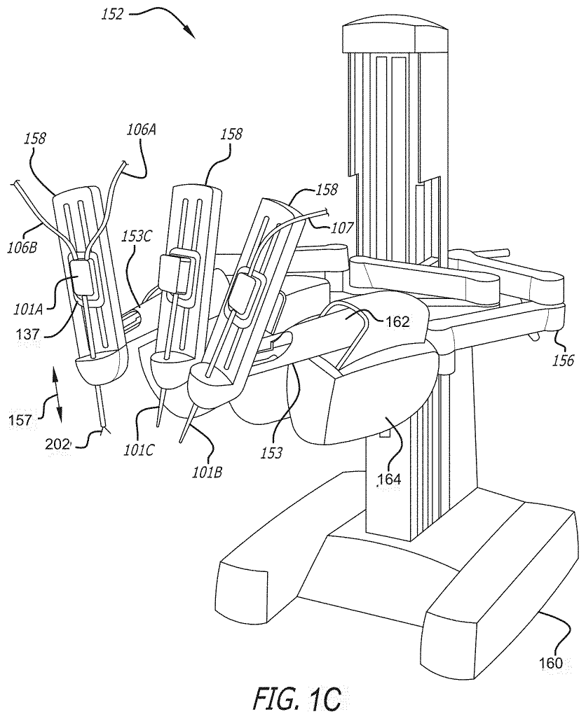

Referring now to FIG. 1A, a block diagram of a teleoperated surgery system 100A is illustrated to perform minimally invasive teleoperated surgical procedures using electrosurgical tools 101A and 101B. Each of the electrosurgical tools 101A and 101B are teleoperated endoscopic surgical instrument that are manipulated by a slaved teleoperated manipulator and remotely controlled by control signals received from a master control console. In contrast, manual endoscopic surgical instruments are directly controlled by hand. Electrosurgical tool 101A is a bipolar electrosurgical tool. Electrosurgical tool 101B is a monopolar electrosurgical tool.

A user or operator O (generally a surgeon) performs a minimally invasive surgical procedure on patient P by manipulating input devices at a master control console 150. The master control console 150 may also be referred to herein as a control console, a surgeon console, or a master console. A computer 151 of the console 150 directs movement of teleoperated endoscopic surgical instruments (generally numbered 101), effecting movement of the instruments using a teleoperated surgical manipulator 152. The teleoperated surgical manipulator 152 may also be referred to as teleoperated patient-side cart system or simply as a cart. The teleoperated surgical manipulator 152 has one or more teleoperated surgical arms 153A-D. Typically, the teleoperated surgical manipulator 152 includes at least three teleoperated surgical arms 153A-D supported by linkages, with a central arm supporting an endoscopic camera 101C and the teleoperated surgical arms 153A-D to left and right of center supporting tissue manipulation tools and the electrosurgical surgical tool 101A.

An assistant A may assist in pre-positioning of the teleoperated surgical manipulator 152 relative to patient P as well as swapping tools or surgical instruments 101 for alternative tool structures, and the like, while viewing the internal surgical site via an assistant's display 154. The image of the internal surgical site shown to A by the assistant's display 154 and operator O by surgeon's console 150 is provided by one of the surgical instruments 101 supported by the teleoperated surgical manipulator 152.

Generally, the teleoperated surgical manipulator 152 include a positioning portion and a driven portion. The positioning portion of the teleoperated surgical manipulator 152 remains in a fixed configuration during surgery while manipulating tissue. The driven portion of the teleoperated surgical manipulator 152 is actively articulated under the direction of the operator O generating control signals at the surgeon's console 150 during surgery. The actively driven portion of the teleoperated surgical arms 153 is herein referred to as an actuating portion 158. The positioning portion of the teleoperated surgical arms 153 that are in a fixed configuration during surgery may be referred to as positioning linkage and/or “set-up joint” 156, 156′.

The surgical instrument interface may further comprise an electrical connector for connecting the conductor to an external electrosurgical generator. The surgeon may activate an input, such as a foot switch, causing the generator to supply electrical energy through a power cord and the conductor to the end effector. Typically a high frequency AC or RF current may be employed, with the voltage being dependent on the type and degree of treatment desired. Voltages may range up to 12,000V in some cases, with about 3000V being a typical value, e.g., for coagulation in monopolar instruments and lower voltages of ˜500V for cutting with bipolar instruments.

The conductor generally provides electrosurgical treatment in a safe and effective manner that minimizes current leakage as the conductor is largely insulated from the tool base to the distal end of the shaft. The invention incorporates a variety of safety features to prevent current leakage to non-target tissue so as to reduce collateral tissue damage, unwanted burning, or the like. Unintended current leakage can be minimized or prevented by insulating the conductor within the elongate shaft and by extending the conductor to the electrode. The area adjacent to the point of contact with the electrode may be potted to prevent current leakage.

To support the functionality of the electrosurgical tools 101A-101B, the teleoperated surgical system 100 may further include one or more electrosurgical generators 102A-102B. The one or more electrosurgical generators 102A-102B are remotely controlled by the master console 150 over the control cables 109A-109B by a surgeon operating the master console.

In one embodiment the electrosurgical generator 102A is a bipolar generator. A pair of wires 106A-106B couple between the bipolar electrosurgical generator 102A and a bipolar electrosurgical tool 101A. The pair of wires 106A-106B may transfer the energy of the bipolar electrosurgical generator 102A to a respective pair of end effectors of the bipolar electrosurgical tool 101A to cauterize, seal, desiccate or cut tissue.

In other embodiments electrosurgical generator 102B is a monopolar generator. A wire 107 couples between the monopolar electrosurgical generator 102B and a monopolar electrosurgical tool 101B. A ground wire 108 couples between the monopolar electrosurgical generator 102B and patient P. The wire 107 may transfer the energy of the monopolar electrosurgical generator 102B to an end effector of the monopolar electrosurgical tool 101B to cauterize or seal tissue. A monopolar electrosurgical generator and a bipolar electrosurgical generator may be combined together into one electrosurgical generator 102A′ that can be remotely controlled by two sets of controls from the control console 150. That is, a first set of controls of the equipment 102A′ can be used to control one function of the remote controlled equipment to supply (e.g., monopolar electrosurgical energy) a first teleoperated surgical tool while a second set of controls of the equipment can be used to control another function of the remote controlled equipment to supply (e.g., bipolar electrosurgical energy) a second surgical tool. The remote controlled equipment may also be referred to as remote controllable equipment or remote controlled supply equipment. The surgical tools that couple to the remote controlled equipment to receive a supply may also be referred to as supply controllable tools.

Referring now to FIG. 1B, a block diagram of a teleoperated surgery system 100B is illustrated. The teleoperated surgery system 100B is similar to the teleoperated surgery system 100A but with a control cart 150B being introduced between the surgeon's console 150A and the patient side cart 152. The control cart 150B includes a computer 151B, and optionally, an external monitor 154. To further control or support the teleoperated surgical tools, the control cart 150B includes one or more pieces of remote controllable equipment 102A′-102N′.

One piece of remote controllable equipment 102A′ mounted in the control cart may be an electrosurgical generator that combines a monopolar electrosurgical generator and a bipolar electrosurgical generator together to supply electrosurgical energy to two electrosurgical tools 101A-101B. A pair of wires 106A-106B couple between the electrosurgical generator 102A′ for a bipolar electrosurgical tool 101A. The pair of wires pair of wires 106A-106B may transfer the energy of the bipolar electrosurgical generator 102A′ to a respective pair of end effectors of the bipolar electrosurgical teleoperated surgical tool 101A to cauterize or seal tissue. A wire 107 couples between the electrosurgical generator 102A′ and a monopolar electrosurgical teleoperated tool 101B. A ground wire 108 (not shown in FIG. 1A, see FIG. 1B) is used to couple between the electrosurgical generator 102A′ and a patient P.

A control cable 110 couples between the computer 151B of the control cart 150B and the surgeon's console to control the surgical system, including the remote controllable equipment and the teleoperated surgical arms and teleoperated surgical tools. A control cable 111 is coupled to the computer 151B and the patient side cart 152 for the surgeon's console to control the teleoperated arms and surgical tools through the control cart.

Smart cables 112A-112N may be respectively coupled between the one or more pieces of remote controllable equipment 102A′-102N′ and the computer 151B in the control cart 150B. With these connections, the surgeon's console can control the remote controllable equipment with its foot pedals and master controllers. In this manner, the control of the remote controllable equipment 102A′-102N′ may be integrated into the surgeon's console. Its foot pedals and master controllers become integrated control mechanisms that a surgeon may use to control every aspect of the surgical system to make teleoperated surgery more efficient. Advanced user interfaces may be used to provide improved control and feedback of operating the remote controllable equipment with the teleoperated surgical tools.

### Patient Side Cart (Teleoperated Surgical Manipulator)

Referring now to FIG. 1C, a perspective view of the teleoperated surgical manipulator 152 is illustrated. The teleoperated surgical manipulator 152 may also be referred to as a patient side cart (PSC).

The teleoperated surgical manipulator 152 has one or more teleoperated surgical arms 153. The teleoperated surgical arm 153C includes an electrosurgical tool 101A coupled thereto. The teleoperated surgical manipulator 152 further includes a base from which the electrosurgical surgical instruments 101 may be supported. More specifically, the teleoperated surgical instruments 101 are each supported by the positioning linkage 156 and the actuating portion 158 of the arms 153. It should be noted that these linkage structures are here illustrated with protective covers 162, 164 extending over much of the teleoperated arms. It should be understood that these protective covers 162, 164 are optional, and may be limited in size or entirely eliminated in some embodiments to minimize the inertia that is manipulated by the servomechanism, and to limit the overall weight of teleoperated surgical manipulator 152.

Each of the surgical tools 101A-101C, releasably couple to a moveable carriage 137 near an end of each teleoperated surgical arm. Each moveable carriage 137, with the teleoperated surgical tool mounted thereto, can be driven to translate along a linear guide formation in the actuating portion 158 of the teleoperated surgical arms 153 in the direction of arrow 157.

The teleoperated surgical manipulator 152 generally has dimensions suitable for transporting between operating rooms. Wheeled base 160 typically can fit through standard operating room doors and onto standard hospital elevators. The teleoperated surgical manipulator 152 may have a weight and a wheel (or other transportation) system that allows the cart to be positioned adjacent an operating table by a single attendant. The teleoperated surgical manipulator 152 may be sufficiently stable during transport to avoid tipping, and to easily withstand overturning moments that may be imposed at the ends of the teleoperated arms during use.

Each of the teleoperated manipulating arms 153 preferably includes a linkage that constrains the movement of the surgical tool 101 mounted thereto. More specifically, linkage includes rigid links coupled together by rotational joints in a parallelogram arrangement so that the teleoperated surgical tools rotate around a point in space. At the point in space, the teleoperated arm can pivot the teleoperated surgical tool about a pitch axis and a yaw axis. The pitch and yaw axes intersect at the point, which is aligned along a shaft of the surgical tool 101. The shaft is a rotatable hollow tube that may have a number of cables of a cable drive system to control the movement of the end effectors 202 mounted at the distal end of the rotatable hollow tube.

The teleoperated arm provides further degrees of freedom of movement to the teleoperated surgical tool. Along an insertion axis, parallel to the central axis of the shaft of the teleoperated surgical tool, the teleoperated surgical tool may slide into and out from a surgical site as indicated by arrow 157. The teleoperated surgical tool can also rotate about the insertion axis. As the teleoperated surgical tool slides along or rotates about the insertion axis, the center point is relatively fixed with respect to the base patient side cart 152. That is, the entire teleoperated arm is generally moved in order to maintain or re-position back to the center point.

The linkage of the teleoperated arm may be driven by a series of motors therein in response to commands from a processor or computer. The motors in the teleoperated arm are also used to rotate and/or pivot the teleoperated surgical tool at the center point around the axes. If a surgical tool 101 further has end effectors to be articulated or actuated, still other motors in the teleoperated arm may be used to control the end effectors. Additionally, the motion provided by the motors may be mechanically transferred to a different location such as by using pulleys, cables, gears, links, cams, cam followers, and the like or other known means of transfer, such as pneumatics, hydraulics, or electronics.

### Teleoperated Electrosurgical Tool

The surgical tools 101 are generally sterile structures, often being sterilizable and/or being provided in hermetically sealed packages for use. As the teleoperated surgical tools 101 will be removed and replaced repeatedly during many procedures, a tool holder could potentially be exposed to contamination if the interface directly engages the tool holder. To avoid contamination to a tool holder and possible cross contamination between patients, an adaptor for coupling to teleoperated surgical tools 101 is provided in a teleoperated arm of the teleoperated surgical manipulator.

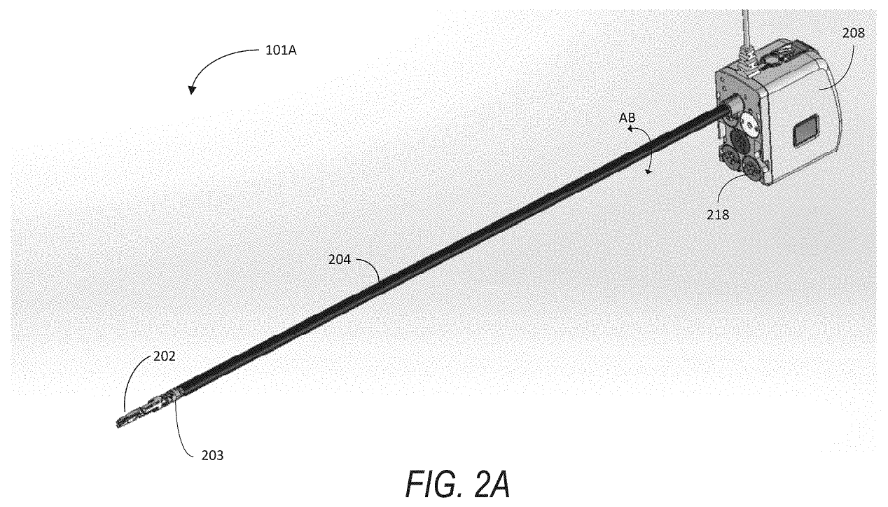

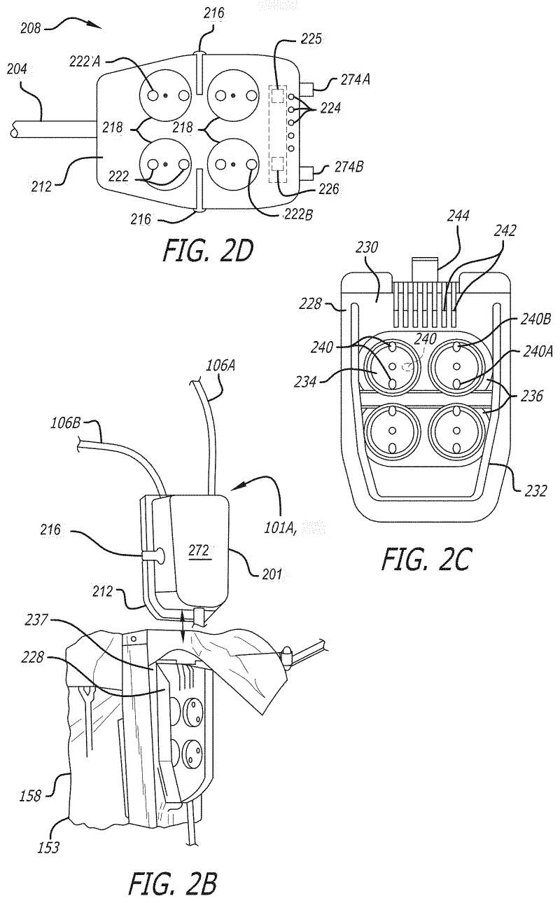

Referring now to the FIG. 2A; a perspective view of an exemplary embodiment of the electrosurgical tool 101A generally including four main sections: a mountable housing 208, a shaft 204, a wrist 203, and an end effector 202. The mountable housing 208 mounts onto an adapter 228 on the teleoperated surgical arm 153. Rotatable receiving members 218 on the mountable housing 201 mechanically couple to rotatable drivers 234 on the teleoperated surgical arm 153. Rotation of the rotatable drivers 234, rotate the rotatable receiving members 218 which in turn actuate rods and/or cables in the shaft 204 to actuate the wrist 203 and/or end effectors 202. A more detailed explanation of the electrosurgical tool 101/101a is given below with reference to FIG. 2B-2D illustrating different views of the mountable housing 208 and adapter 228 of the teleoperated surgical arm 153.

Referring now to FIGS. 2B-2D, the mounting of the electrosurgical tool 101A to an adapter 228 of the teleoperated surgical arm is now briefly described. The teleoperated surgical arm 153 may include an adapter 228 to which the electrosurgical tool 101A or other surgical tool 101 may be mounted. FIG. 2C illustrates a front side of an exemplary adapter 228. The front side of the adaptor 228 is generally referred to as a tool side 230 and the opposite side is generally referred to as a holder side (not shown).

FIG. 2D illustrates a back side of an exemplary electrosurgical tool 101A. The electrosurgical tool 101A includes an exemplary mountable housing 208 including an interface base 212 that can be coupled to the adapter 228 to mount the electrosurgical tool 101A to a teleoperated arm of a teleoperated surgical manipulator. The interface base 212 and the adapter 228 may be electrically and mechanically coupled together to actuate the electrosurgical tool 101A. Rotatably coupled to the interface base 212 are one or more rotatable receiving members 218, also referred to as input disks. Each of the one or more rotatable receiving members 218 includes a pair of pins 222A and 222B generally referred to as pins 222. Pin 222A is located closer to the center of each rotatable receive member 218 than pin 222B. The one or more rotatable receiving members 218 can mechanically couple respectively to one or more rotatable drivers 234 of the adapter 228. The electrosurgical tool 101A may further include release levers 216 to release it from the adapter 228 and the teleoperated arm.

The interface base 212 may further include one or more electrical contacts or pins 224 to electrically couple to terminals of an electrical connector 242 of the adapter 228. One or more terminals of the electrical connector 242 that can couple to the electrical contacts or pins 224 of the tool may be used to make electrocautery connections, such as between an integrated controller and the tool and/or between the tool and electrosurgical generating units. The interface base 212 may further include a printed circuit board 225 and one or more integrated circuits 226 coupled thereto and to the one or more pins 224. The one or more integrated circuits 226 store tool information that may be used to identify the type of teleoperated surgical tool coupled to the teleoperated arm, so that it may be properly controlled by the master control console 150.

Referring to FIGS. 2B and 2D, an electrosurgical tool or instrument 101A is illustrated. The electrosurgical tool 101A includes a mountable housing 208, an elongated shaft 204 having a proximal end and a distal end; and end effectors (not shown) coupled near the distal end of the shaft 204. The mountable housing 208 includes an interface or tool base 212 coupled to the proximal end of the shaft 204. The mountable housing 208 may further include one or more electrical connectors 274A-274B, a cover 272, and one or more release levers 216. At the distal end of the shaft 204, a mechanical wrist (not shown) may be used to move the end effectors.

The interface or tool base 212 of the electrosurgical tool 101A can couple to an adapter 228 so that it is removeably connectable to the teleoperated surgical system. Other surgical tools with the same type of tool base may also couple to the adapter and on the teleoperated arm. During surgery, the adapter 228 is coupled to the moveable carriage 237. Thus, with the electrosurgical tool 101A mounted to the adapter 228, it can translate with the carriage 237 along an insertion axis of the teleoperated surgical arm 153 as indicated by arrow 157 in FIG. 1C. The tool base 212 includes receiving elements or input disks 218 that releasably couple through the adapter 228 to a rotatable driving element 234 that is mounted on the carriage 237 of the teleoperated arm assembly 153. The rotatable driving elements 234 of the carriage 237 are generally coupled to actuators (not shown), such as electric motors or the like, to cause selective angular displacement of each in the carriage 237.

When mounted to a teleoperated surgical arm 153, end effectors 202 may have a plurality of degrees of freedom of movement relative to arm 153, in addition to actuation movement of the end effectors. The end effectors of the teleoperated surgical tool are used in performing a surgical operation such as cutting, shearing, grasping, gripping, clamping, engaging, or contacting tissue adjacent a surgical site. With an electrosurgical tool 101A, a conductor electrically communicates with the end effector to deliver electrical energy to tissue clamped by the gripping jaws or otherwise in contact with the end effector.

As shown in FIG. 2D, the tool base 212 may be enclosed by a cover 272 to which one or more electrical connectors 274A-274B may be mounted. The one or more electrical connectors 274A-274B can receive one or more cables 106A-106B to couple to an electrosurgical generator unit, such as the bipolar generator 102A, the monopolar generator 102B, or a combined monopolar/bipolar generator 102A′ illustrated in FIG. 1A. One or more wires within the tools electrically couple between the electrical connectors 274A-274B and the one or more electrodes at the end effector of the tool. Alternatively, one or more terminals 242 of the electrical connector 274A-B that can couple to the electrical contacts or pins 224 of the tool may be used to make the electrocautery connections between the tool and the electrosurgical generating units.

The adapter 228 includes one or more rotatable drivers 234 rotatably coupled to a floating plate 236. The rotatable drivers 234 are resiliently mounted to the floating plate 236 by resilient radial members which extend into a circumferential indentation about the rotatable drivers. The rotatable drivers 234 can move axially relative to floating plate 236 by deflection of these resilient structures.

The floating plate 236 has a limited range of movement relative to the surrounding adaptor structure normal to the major surfaces of the adaptor. Axial movement of the floating plate helps decouple the rotatable drivers 234 from an electrosurgical tool 101A when its release levers 216 are actuated.

The one or more rotatable drivers 234 of the adapter 228 may mechanically couple to a part of the surgical tools 101. Each of the rotatable drivers 234 may include one or more openings 240 to receive protrusions or pins 222 of rotatable receiving members 218 of the surgical tools 101. The openings 240 in the rotatable drivers 234 are configured to accurately align with the rotatable receiving elements 218 of the surgical tools 101. In other embodiments of the invention, pins 222 and rotatable receiving members 218 may be swapped. In such embodiments, the pins 222 would be on the rotatable drivers 234 and the openings 240 would be on rotatable receiving members 218.

The inner pins 222A and the outer pins 222B of the rotatable receiving elements 218 respectively align with the opening 240A and the opening 240B in each rotatable driver. The pins 222A and openings 240A are at differing distances from the axis of rotation than the pins 222B and openings 240B so as to ensure that rotatable drivers 234 and the rotatable receiving elements 218 are not aligned 180 degrees out of phase from their intended position. Additionally, each of the openings 240 in the rotatable drivers may be slightly radially elongated so as to fittingly receive the pins in the circumferential orientation. This allows the pins 222 to slide radially within the openings 240 and accommodate some axial misalignment between the tool and the adapter 228, while minimizing any angular misalignment and backlash between the rotatable drivers 234 and the rotatable receiving elements 218. Additionally, the interaction between pins 222 and openings 240 helps restrain the electrosurgical tool 101A in the engaged position with the adapter 228 until the release levers 216 along the sides of the housing 208 push on the floating plate 236 axially from the interface so as to release the tool 101.

When disposed in a first axial position (away from the tool side 230) the rotatable drivers are free to rotate without angular limitation. The one or more rotatable drivers 234 may rotate clockwise or counter-clockwise to further actuate the systems and tools of the teleoperated surgical instruments 101. However, as the rotatable drivers move axially toward the tool side 230, tabs (extending radially from the rotatable drivers) may laterally engage detents on the floating plates so as to limit the angular rotation of the rotatable drivers about their axes. This limited rotation can be used to help engage the rotatable drivers the rotating members of the tool as the pins 222 may push the rotatable bodies into the limited rotation position until the pins are aligned with (and slide into) the openings 240 in the rotatable drivers.

While rotatable drivers 234 are described here, other types of drivers or actuators may be provided in the adapter 228 to actuate systems or tools of the teleoperated surgical instruments 101. The adapter 228 further includes terminals of an electrical connector 242 to couple to electrical contacts or pins 424 of surgical instruments 101 to make an electrical connection as well.

The mounting of electrosurgical tool 101A to the adapter 228 generally includes inserting the tip or distal end of the shaft or hollow tube of the teleoperated surgical tool through a cannula (not shown) and sliding the interface base 212 into engagement with the adapter 228, as illustrated in FIG. 2C. A lip 232 on the tool side 230 of the adaptor 228 slideably receives the laterally extending portions of the interface base 212 of the teleoperated surgical tool. A catch 244 of adapter 228 may latch onto the back end of the interface base 212 to hold the tool 101A in position. The protrusions or pins 222 extending from the one or more rotatable receiving elements 218 of the teleoperated surgical tool couple into the holes 240A-240B (generally referred to as holes or openings 240) in the rotatable drivers 234 of the adapter 228.

The range of motion of the rotatable receiving elements 218 in the teleoperated surgical tool may be limited. To complete the mechanical coupling between the rotatable drivers of the adapter and the rotatable receiving elements 218, the operator O at the surgical master control console 150 may turn the rotatable drivers in one direction from center, turn the rotatable drivers in a second direction opposite the first, and then return the rotatable drivers to center. Further, to ensure that the pins 222 enter openings 240 of rotatable drivers adapter 228, the adapter 228 and tool 101A mounted thereto may be moved together. The adapter 228 and tool 101A mounted thereto may be moved to an initial position so that the tip or distal end of the shaft or hollow tube is disposed within a cannula (not shown).

To dismount and remove the electrosurgical tool 101A, the release levers 216 may be squeezed pushing out on the mountable housing 208 to release the pins 222 from the holes 240 and the catch 244 from the back end of the interface base. The mountable housing 208 is then pulled up to slide the interface base 212 up and out from the adapter 228. The mountable housing 208 is continually pulled up to remove the tip or distal end of the shaft or hollow tube out from the cannula 219. After the electrosurgical tool 101A is dismounted, another teleoperated surgical tool may be mounted in its place, including a new or freshly sterilized electrosurgical tool 101A.

As previously discussed, the electrosurgical tool 101 may include one or more integrated circuits 226 to identify the type of teleoperated surgical tool coupled to the teleoperated arm, such that it may be properly controlled by the master control console 150. However, the teleoperated surgical system may determine whether or not the teleoperated surgical tool is compatible or not, prior to its use.

The system verifies that the tool is of the type which may be used with the teleoperated surgical system 100. The one or more integrated circuits 226 may signal to the computer 151 in the master control console 150 data regarding compatibility and tool-type to determine compatibility as well as control information. One of the integrated circuits 226 may include a non-volatile memory to store and read out data regarding system compatibility, the tool-type and the control information. In an exemplary embodiment, the data read from the memory includes a character string indicating tool compatibility with the teleoperated surgical system 100. Additionally, the data from the tool memory will often include a tool-type to signal to the master control console how it is to be controlled. In some cases, the data will also include tool calibration information. The data may be provided in response to a request signal from the computer 151.

Tool-type data will generally indicate what kind of tool has been attached in a tool change operation. The tool-type data may include information on wrist axis geometries, tool strengths, grip force, the range of motion of each joint, singularities in the joint motion space, the maximum force to be applied via the rotatable receiving elements, the tool transmission system characteristics including information regarding the coupling of rotatable receiving elements to actuation or articulation of a system within the teleoperated surgical instrument.

For example, the tool-type data might indicate that an electrosurgical instrument 101A has been mounted to the teleoperated arm or not. Relevant to energy activation of an electrosurgical instrument, additional tool type data related to primary and/or secondary energy sub-features may further be stored. For example, energy sub-features may include what type of electrosurgical energy the tool may receive (e.g., bipolar or monopolar cutting & monopolar coagulating), maximum peak energy, minimum harmonic energy frequency, maximum harmonic energy frequency, and whether or not a laser is also provided for cutting. As new energy or other types of modalities are introduced for teleoperated surgical tools, its tool-type data can be readily stored and communicated to the teleoperated surgical system so that the system can adaptively control remote controllable equipment and multiple types of teleoperated surgical tools mounted to teleoperated arms of the teleoperated surgical system.

Instead of storing all of the tool-type data in the one or more integrated circuits 426, most of the tool-type data may optionally be stored in memory or a hard drive of the computer 151 in the teleoperated surgical system 100. An identifier may be stored in the one or more integrated circuits 226 to signal the computer 151 to read the relevant portions of data in a look up table store in the memory or the hard drive of the computer. The tool-type data in the look-up table may be loaded into a memory of computer 151 by the manufacturer of the teleoperated surgical system 100. The look-up table may be stored in a flash memory, EEPROM, or other type of non-volatile memory. As a new tool-type is provided, the manufacturer can revise the look-up table to accommodate the new tool-specific information. It should be recognized that the use of tools which are not compatible with the teleoperated surgery system, for example, which do not have the appropriate tool-type data in an information table, could result in inadequate control over the teleoperated surgical tool by the computer 151 and the operator O.

In addition to the tool-type data, tool specific information may be stored in the integrated circuit 226, such as for reconfiguring the programming of computer 151 to control the tool. There may be calibration information, such an offset, to correct a misalignment in the teleoperated surgical tool. The calibration information may be factored into the overall control of the teleoperated surgical tool. The storing of such calibration information can be used to overcome minor mechanical inconsistencies between tools of a single type. For example, the tool-type data including the tool-specific data may be used to generate appropriate coordinate transformations and servo drive signals to manipulate the teleoperated arm and rotate the rotatable drivers 234. In this case, the integrated circuit 226 includes the information to set up the control system to drive the end effectors in the tool to have a maximum joint torque setting so that the jaws of a robotic gripping tool or a electrosurgical tool can clamp to tissue with a maximum force.

Additionally, some teleoperated surgical tools have a limited life span. Tool life and cumulative tool use information may also be stored on the tool memory and used by the computer to determine if the tool is still safe for use. Total tool life may be measured by clock time, by procedure, by the number of times the tool has been loaded onto a holder, and in other ways specific to the type of tool. Tool life data is preferably stored in the memory of the tool using an irreversible writing process.

### Surgical Master Control Console

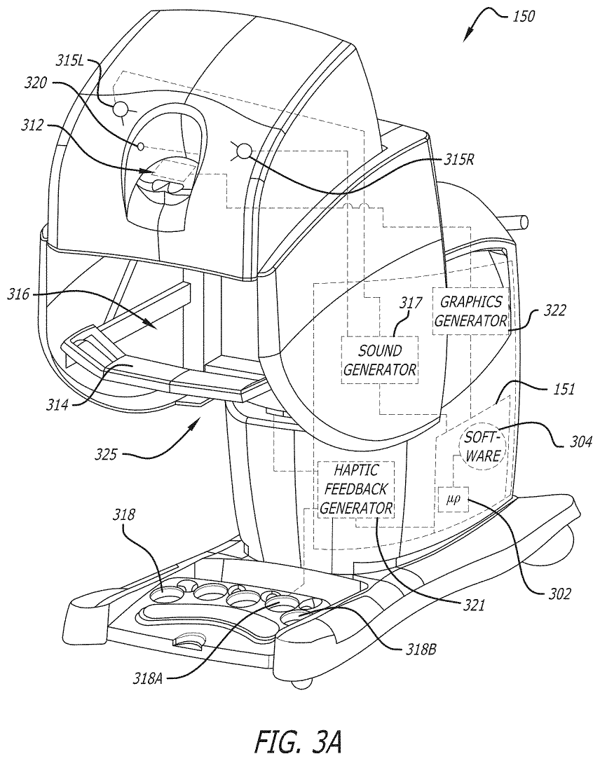

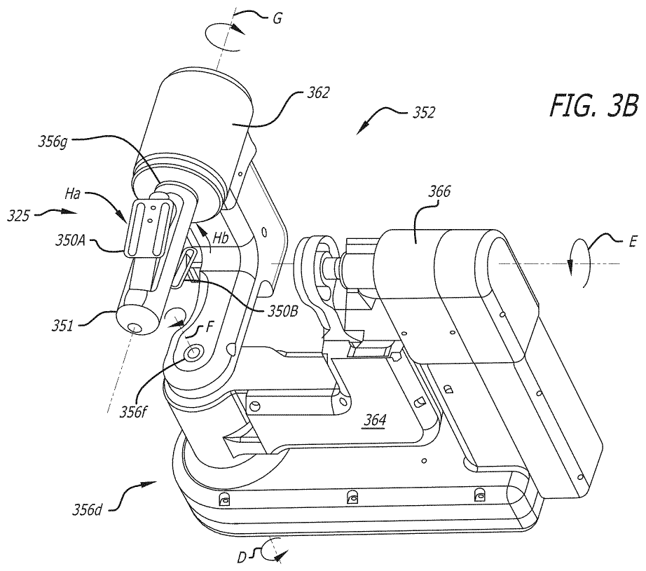

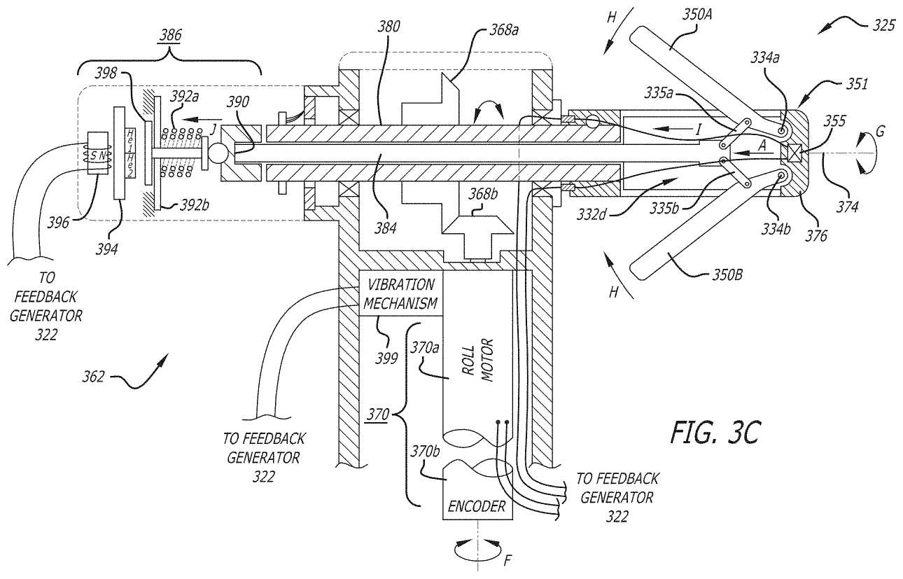

Referring now to FIG. 3A, a perspective view of a teleoperated surgical master control console 150 is illustrated. The master control console 150 of the teleoperated surgical system 100 includes the computer 151, a binocular viewer 312, an arm support 314, a microphone 315, a pair of control input wrists and control input arms in a workspace 316, a speech recognizer 317, foot pedals 318 (including foot pedals 318A-318B), and a viewing sensor 320.

The computer 151 may include one or microprocessors 302 to execute instructions and a storage device 304 to store software with executable instructions that may be used to generate control signals to control the teleoperated surgical system 100. The master control console 150 generates the control signals to control the electrosurgical instruments in a surgical site.

The viewer 312 has at least one display where images of a surgical site may be viewed to perform minimally invasive surgery.

The arm support 314 can be used to rest the elbows or forearms of the operator O (typically a surgeon) while gripping touch sensitive handles 325 (see FIGS. 3B-3C), one in each hand, of the pair of control input wrists 352 in the workspace 316 to generate control signals. The touch sensitive handles 325 are positioned in the workspace 316 disposed beyond the arm support 314 and below the viewer 312.

When using the master control console, the operator O typically sits in a chair, moves his or her head into alignment with the binocular viewer 312, and grips the touch sensitive handles 325 of the control input wrists 352, one in each hand, while resting their forearms against the arm support 314. This allows the touch sensitive handles to be moved easily in the control space 316 in both position and orientation to generate control signals.

Additionally, the operator O can use his feet to control the foot-pedals to change the configuration of the surgical system and generate additional control signals to control teleoperated surgical instruments.

To ensure that the operator is viewing the surgical site when controlling the surgical tools 101, the master control console 150 may include the viewing sensor 320 disposed adjacent the binocular display 312. When the system operator aligns his or her eyes with the binocular eye pieces of the display 312 to view a stereoscopic image of the surgical worksite, the operator's head sets off the viewing sensor 320 to enable the control of the surgical tools 101. When the operator's head is removed the area of the display 312, the viewing sensor 320 can disable or stop generating new control signals in response to movements of the touch sensitive handles in order to hold the state of the surgical tools.

The computer 151 with its microprocessors 302 interprets movements and actuation of the touch sensitive handles 325 (and other inputs from the operator O or other personnel) to generate control signals to control the surgical instruments 101 in the surgical worksite. In one embodiment of the invention, the computer 151 and the viewer 312 map the surgical worksite into the controller workspace 316 so it feels and appears to the operator that the touch sensitive handles 325 are working over surgical worksite.

Referring now to FIG. 3B, a perspective view of a control input wrist 352 with a touch sensitive handle 325 is illustrated. The control input wrist 352 is a gimbaled device that pivotally supports the touch sensitive handle 325 of the master control console 150 to generate control signals that are used to control the teleoperated surgical manipulator 152 and the surgical tools 101, including electrosurgical tool 101A,101C. A pair of control input wrists 352 are supported by a pair of control input arms in the workspace 316 of the master control console 150.

The control input wrist 352 includes first, second, and third gimbal members 362, 364, and 366. The third gimbal member is rotationally mounted to a control input arm (not shown).

The touch sensitive handle 325 includes a tubular support structure 351, a first grip 350A, and a second grip 350B. The first grip and the second grip are supported at one end by the structure 351. The touch sensitive handle 325 can be rotated about axis G illustrated in FIGS. 3B-3C. The grips 350A, 350B can be squeezed or pinched together about the tubular structure 351. The “pinching” or grasping degree of freedom in the grips is indicated by arrows Ha,Hb in FIG. 3B and arrows H in FIG. 3C.

The touch sensitive handle 325 is rotatably supported by the first gimbal member 362 by means of a rotational joint 356g. The first gimbal member 362 is in turn, rotatably supported by the second gimbal member 364 by means of the rotational joint 356f. Similarly, the second gimbal member 364 is rotatably supported by the third gimbal member 366 using a rotational joint 356d. In this manner, the control wrist allows the touch sensitive handle 325 to be moved and oriented in the workspace 316 using three degrees of freedom.

The movements in the gimbals of the control wrist 352 to reorient the touch sensitive handle in space can be translated into control signals to control the teleoperated surgical manipulator 152 and the surgical tools 101.

The movements in the grips 350A,350B of the touch sensitive handle 325 can also be translated into control signals to control the teleoperated surgical manipulator 152 and the surgical tools 101. In particular, the squeezing motion of the master grips 350A,350B over their freedom of movement indicated by arrows Ha,Hb or H, may be used to control the end effectors of the surgical tools.

To sense the movements in the touch sensitive handle 325 and generate controls signals, sensors can be mounted in the handle 325 as well as the gimbal member 362 of the control input wrist 352. Exemplary sensors may be a Hall effect transducer, a potentiometer, an encoder, or the like.

Referring now to FIG. 3C, a cross-sectional view of the touch sensitive handle 325 and gimbal member 362 of the control input wrist 352 is illustrated. FIG. 3C provides an example as to how the touch sensitive handle 325 can be mounted to the control input wrist 352 to sense the gripping and rotation of the handle to control surgical tools 101.

As illustrated in FIG. 3C, the exemplary gimbal member 362 includes beveled gears 368a, 368b which can couple the rotational motion of the touch sensitive handle 325 to a roll sensor 370. The roll sensor 370 may use a potentiometer or encoder 370b included in a roll motor 370a to sense the rotation. Alternatively, a separate roll sensor, such as a potentiometer, may be directly coupled to the shaft 380 to sense the rotation of the touch sensitive handle. In any case, a roll sensor senses the roll motion of the touch sensitive handle 325 and generates control signals in response thereto to control the surgical tools 101.

To sense a squeezing motion in the grips 350A,350B of the touch sensitive handle 325, a remote sensing assembly 386 may be included by the gimbal member 362. The first and second grips 350A,350B are adapted to be squeezed together by a hand of an operator O so as to define a variable grip separation. The grip separation may be determined as a function of a variable grip angle with an axis or as a function of a variable grip separation distance, or the like. Alternative handle actuations, such as movement of a thumbwheel or knob may also be provided in the handle to control the surgical instruments 101.

In the exemplary embodiment, the remote sensor assembly 386 includes a circuit board 394 on which a first and a second Hall effect sensors, HE1, HE2 are mounted. A magnet 396 is disposed distally beyond the circuit board 394 and the Hall effect sensors. A magnetic mass 398 is axially coupled to the proximally oriented surface 390 of a push rod 84. Thus, the magnetic mass 398 moves (as shown by Arrow J) with the push rod 384 and varies the magnetic field at the Hall effect sensors in response actuation of the grips 350A,350B.

To translate the squeezing action of the grips 350A,350B to the sensor 386, the gimbal member 362 includes a push rod 384 within the tubular handle structure 351. Each of the grips 350A, 350B pivot about a respective pivot 334a, 334b in the tubular handle structure 351. Urging links 335a, 335b respectively couple between the grips 350A,350B and a first end of the push rod 384. The squeezing action of the grips 350A,350B is translated into a linear motion on the push rod 384 by means of urging links 335a,335b as shown by arrow A in FIG. 3C. A second end of the push rod 384 couples to the sensor 386. As discussed previously, the magnetic mass 398 is axially coupled to the surface 390 of the push rod 384 in order to sense the linear motion in the push rod and the squeezing motion of the grips 350A,350B.

A biasing mechanism such as spring 392 applies a force against the squeezing motion of the grips to return them to full open when the grips are released. The biasing spring 392 may be a linear or non-linear elastic device biasing against the depression of grips 350A, 350B, e.g., a single or multiple element assembly including springs or other elastic members. For example, spring 392 may comprise a concentric dual spring assembly whereby one spring provides a “softer” bias response as the grips 350A, 350B are initially depressed, and a second spring provides a superimposed “firm” bias response as the grips 350A, 350B approach a fully depressed state. Such a non-linear bias may provide a pseudo force-feedback to the operator.

It should be noted that a wide variety of alternative sensing arrangements may be used to translate the mechanical actuation of the touch sensitive handle and control input wrist into control signals. While Hall effect sensors are included in the exemplary embodiment, alternative embodiments may include encoders, potentiometers, or a variety of alternative optical, electrical, magnetic, or other sensing structures.

### Electrosurgical End Effector

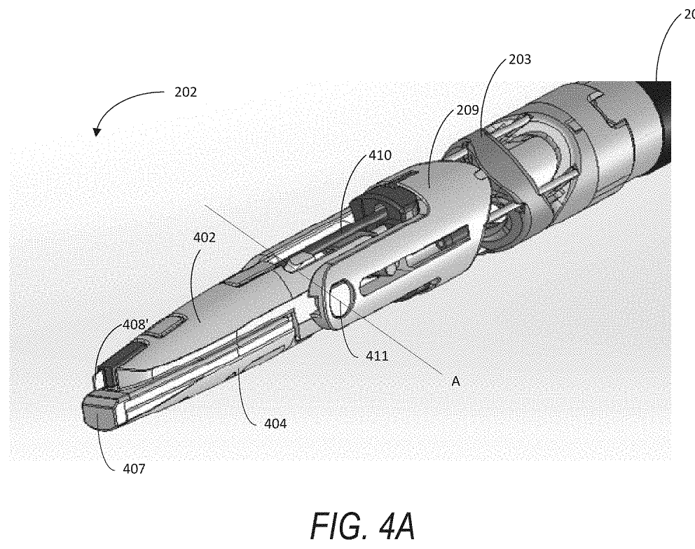

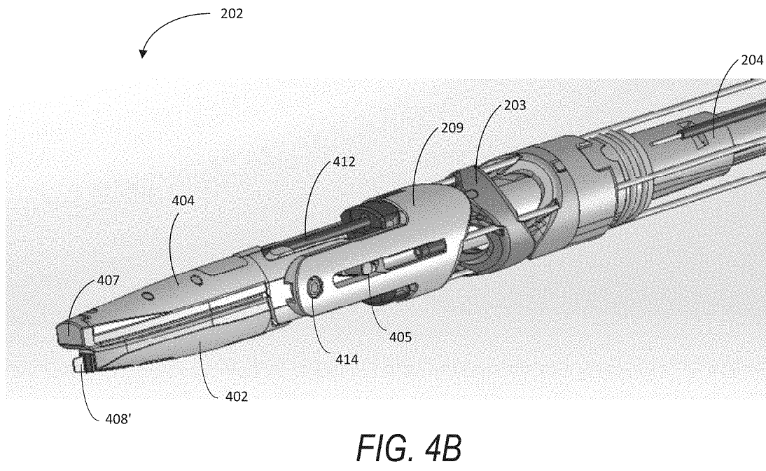

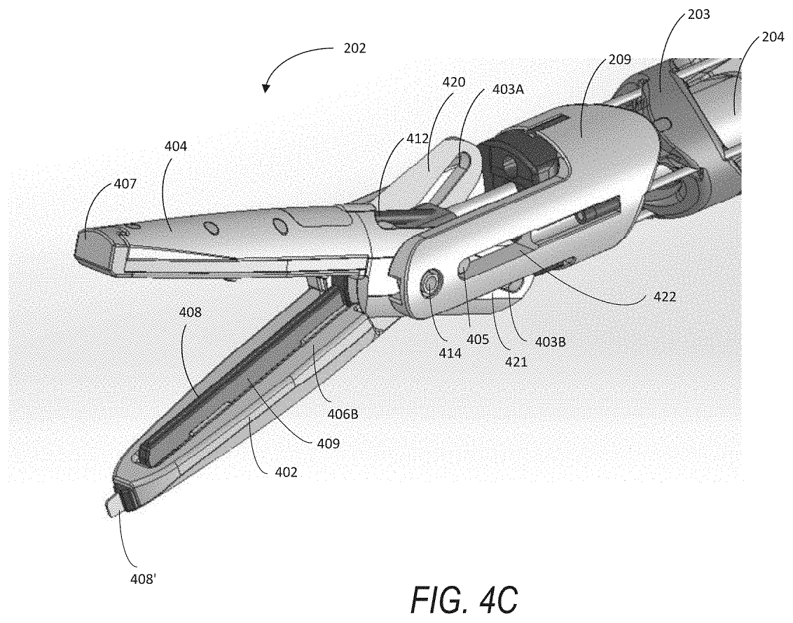

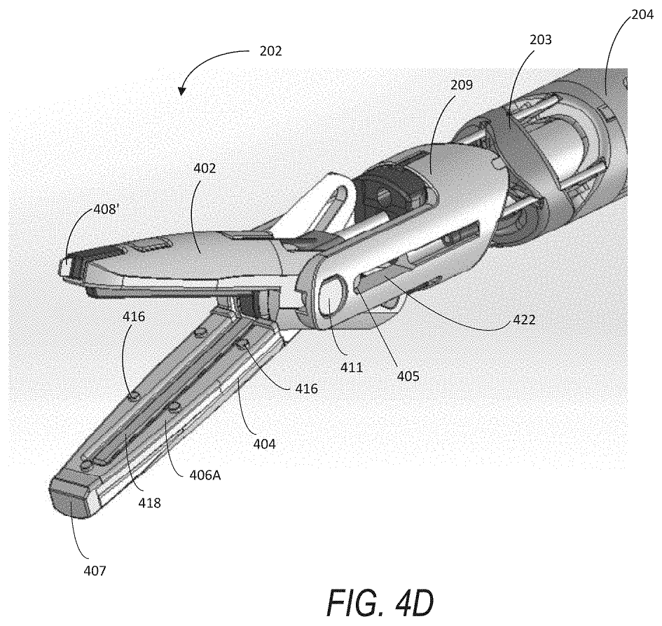

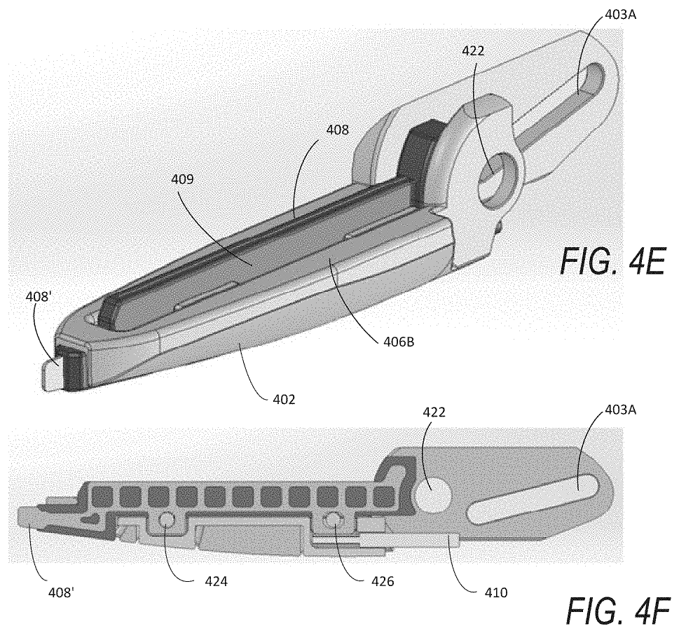

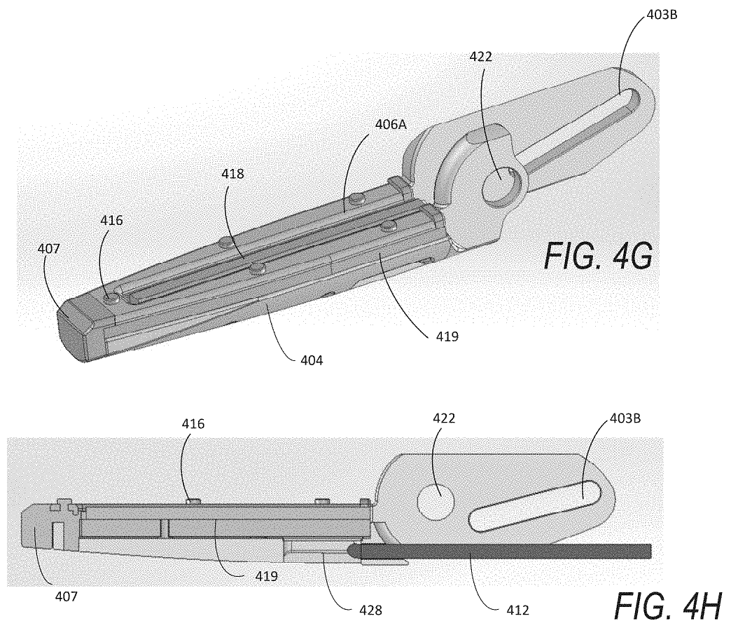

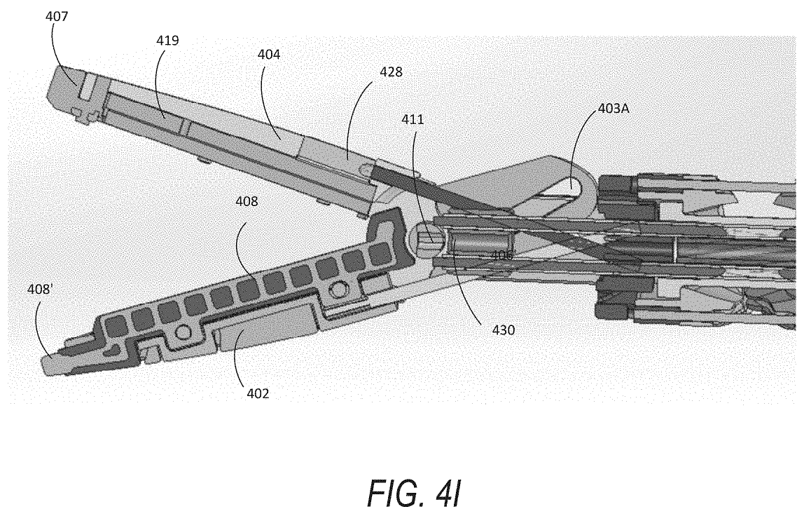

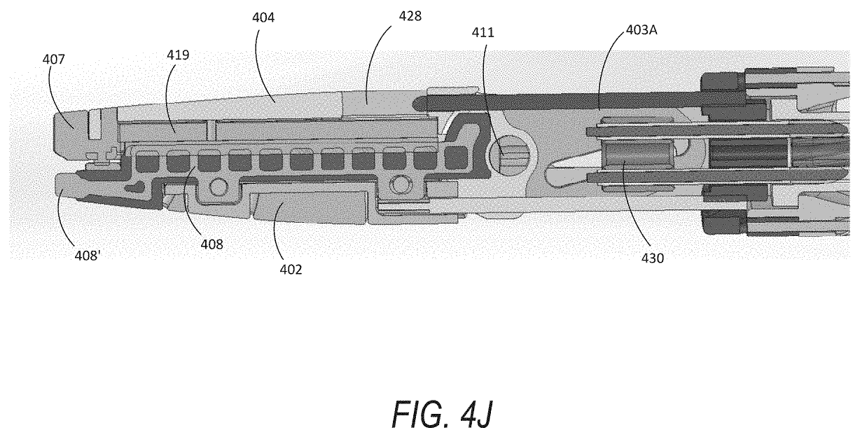

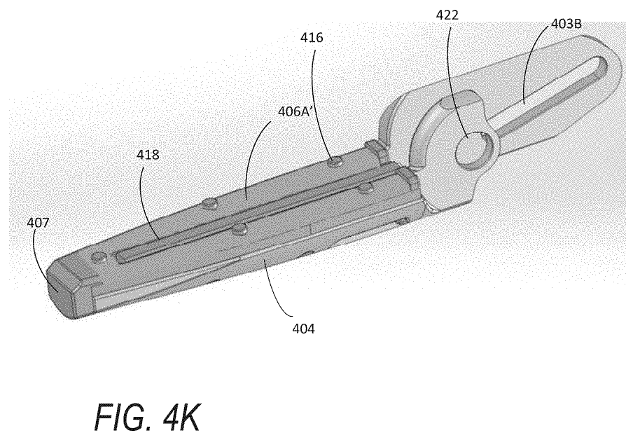

At the distal end of the electrosurgical tool 101A is a surgical end effector 202. The surgical end effector 202 may be one, or in some cases a combination, of a variety of surgical tools including, tissue graspers, needle drivers, scissors, cauterizers, etc. In the present invention, the exemplary surgical end effector illustrated in FIG. 4A-4K is an electrosurgical end effector. An embodiment of the surgical end effector 202 illustrated in FIG. 4A-4K is a combination tissue cutter and sealer that simultaneously delivers electrical energy to separate sealing and cutting electrodes. Other embodiments of the invention may describe surgical end effector 202 with a mechanical knife and compliant sealing electrodes or a bipolar grasper with compliant sealing electrodes without a knife.

Briefly referring back to referring now to FIG. 2A, a surgical instrument 101 for use with the minimally invasive teleoperated surgical system of FIG. 1 comprises an elongate shaft 404 having a proximal end and a distal end. An interface or tool base 201 is coupled to the proximal end of the shaft and removably connectable to the teleoperated surgical system. The interface base 201 may contain receiving members 218 to couple to drivers 234 on the teleoperated arm 153. An end effector 202, for performing a surgical operation such as cutting, shearing, grasping, engaging, or contacting tissue in a surgical site, is mounted at the distal end of the shaft. The drivers 234 provide actuating force to move the end effector 202. In the present invention, the end effector 202 comprises a pair of jaws for cooperatively grasping, sealing, and/or shearing tissue. A conductor electrically communicating with at least one jaw delivers electrical energy to tissue engaged by the jaws or contacting the jaw(s).

At the distal end of the shaft 204 is a mechanical wrist 203 to move the end effectors 202. The interface or tool base 208 can couple to an adapter 228 to which other surgical tools may also couple so that the electrosurgical tool 101A is removably connectable to the teleoperated surgical system. The adapter 228 is coupled to an actuating portion of the teleoperated surgical arm 153. One or more rotatable receiving members 218 on the electrosurgical tool 101A mechanically couple to one or more rotatable drivers 234 of the adapter 228.

When mounted to a teleoperated surgical arm 153, end effectors 202 may have a plurality of degrees of freedom of movement relative to arm 153, in addition to actuation of the end effectors 202. Degrees of freedom of the electrosurgical tool 101A may be provided by an articulating wrist 203 between the shaft 204 and end effector 202. The elongated shaft 204 is rotatably mounted to the base 208 for rotation about an axis extending longitudinally along the shaft 204 as indicated by the rotational arrow AB.

The wrist 203 may be a single pivot wrist, a multi-pivot wrist, a distal roll joint mechanism, or other joints or wrist-like mechanism to provide additional operational degrees of freedom to the end effector. The orientation of the mechanical wrist 203 is controlled through pulleys in the tool base 208 and the wrist 203 with cables of cable loops wrapped around each pulley being routed through the shaft 204. The teleoperated system causes the pulleys in the tool base 208 to be rotated in order to control the position of the mechanical wrist 203. Thus, the cable of the cable loops may also be referred to as a control cable.

Further details of mechanical wrists that may be applicable to the mechanical wrist 203 are described in U.S. patents with filing dates and named inventor as follows U.S. Pat. No. 5,792,135, May 16, 1997, Madhani et al; U.S. Pat. No. 5,979,900, May 16, 1997, Madhani et al; U.S. Pat. No. 5,807,377, May 16, 1997, Madhani et al; U.S. Pat. No. 6,206,903, Oct. 8, 1999, Ramans; U.S. Pat. No. 6,312,435, Oct. 8, 1999, Wallace et al.; U.S. Pat. No. 6,371,952, Jun. 28, 1999, Madhani et al; U.S. Pat. No. 6,394,998, Sep. 17, 1999, Wallace et al.; U.S. Pat. No. 6,676,684, Sep. 4, 2001, Morley et al.; U.S. Pat. No. 6,685,698, Jan. 10, 2003, Morley et al.; U.S. Pat. No. 6,699,235, Mar. 2, 2004, Wallace et al.; U.S. Pat. No. 6,746,443, Jul. 27, 2000, Morley et al.; and U.S. Pat. No. 6,817,974, Jun. 28, 2002, Cooper et al., all of which are incorporated herein by reference.

FIG. 4A-4K provide further details of an exemplary embodiment of an end effector 202. The end effector 202 illustrated in FIG. 4A-4K is an electrosurgical end effector with a cutting tip, a cut electrode, a seal electrode, and one or more elastomeric layers. The electrosurgical end effector 202 may be used in telesurgical operations such as cautery, automy, desiccation, tissue cutting, vessel cutting, and vessel sealing. One of the many advantages of the exemplary electrosurgical end effector is simultaneous tissue cutting and sealing. Another advantage over prior art, is the ability of the electrosurgical end effector 202 to maintain improved tissue contact with the cut electrode due to a novel elastomeric layer in the electrosurgical end effector 202.

When sealing vessels it is important to maintain consistent pressure across the sealing surface of the instrument. To maintain consistent pressure tight engineering tolerances are usually required of the surgical jaws, leading to higher cost and lower volume of manufacturing. Because of the tight tolerances, traditional electrosurgical jaws are expensive to produce and have a high rejection and scrap rate.

With reference to FIG. 4A, a perspective view of an exemplary embodiment of end effector 202. In this embodiment end effector 202 is an electrosurgical end effector illustrated with a cutting tip, a cut electrode, and a seal electrode. Embodiments of the exemplary end effector 202 also comprise an elastomeric layer which aid in cutting and sealing tissue as well as reducing manufacturing cost by allowing greater manufacturing tolerance. Other combinations comprising one or more aforementioned elements are also within the scope of the invention. For example the end effector 202 may comprise a cutting tip, a seal electrode, and an elastomeric layer or a cutting tip and cut electrode with an elastomeric layer, or simply a seal electrode with an elastomeric layer but without a cut electrode, etc.

At the distal end of electrosurgical end effector 202 are twin jaws 402 and jaw 404. In the exemplary illustrations in FIG. 4A-4K, jaws 402 and 404 are substantially cylindrical and slightly tapered at the distal end. Jaws 402 and 404 are rotatable at a pivot axis A through a pivot pin 411. Alternatively, pivot pin 411 may be substituted for other types of fasteners such as a rivet or a bolt and nut that couples jaw 402 and 404 together. In addition to coupling jaws 402 and 404 together, pivot pin 411 also serve to couple the jaws to a clevis 209 mounted to the articulating wrist 203 at the distal end of shaft 204. Jaws 402 and 404 actuate by cooperatively rotating open and close about pivot axis A and pivot pin 411.

Jaw 402 further comprises a cutting tip 408′ and an electrical conductor 410. Electrical conductor 410 may be an insulated wire electrically coupled to an electrical generator 102A,102B. RF energy is conducted from generator 102A,102B through electrical energy conductor 410 to electrodes on jaw 402. Cutting tip 408′ is an electrode at the distal end of jaw 402. Cutting tip 408′ may be used in various surgical procedures such as for perforating an organ wall. When activated, electric current travels from cutting tip 408′ through contacted tissue and returns through the conductive body of jaw 402. In some embodiments of the invention, electrical conductor 410 provides power to both the cut electrode and cutting tip 408′.

Jaw 404 may further comprise a blunt end cap 407 made of an insulative material suitable for surgical conditions. The end cap 407 may aid in preventing accidental discharge of energy into the surgical site if arcing occurs between the cutting tip 408′ and seal electrode 406A.

Referring now to FIG. 4B, a perspective view of an exemplary end effector 202. In FIG. 4B the end effector 202 of FIG. 4A is rotated 180 degrees to show the underside structures of jaw 404. Many of the same structures illustrated in FIG. 4A are again shown in FIG. 4B. Certain additional structures are now visible as well. In this view, a second electrical conductor 412 may be seen conducting energy to jaw 404. Electrical conductor 412 may provide energy to the seal electrodes located on jaw 404. Pivot pin 411 may be swaged at end 414 to hold the pivot pin 411 in place. Alternatively, swaged end 414 may be replaced with a cap or nut 414 to hold pivot pin 411 in place. Additionally, a sliding actuation pin 405 is partially visible. Actuation pin 405 is part of the actuation assembly that causes jaws 402 and 404 to cooperatively rotate open and close. Details of the actuation assembly will be given below.

Moving on to FIG. 4C, a perspective view of end effector 202 in which the end effector is illustrated with jaws 402 and 404 rotated to an open position. In this open position, the working surface of jaw 402 may be seen. The working surface of jaw 402 may comprise two electrodes; a substantially flat seal electrode 406B and a raised cut electrode 408. In the exemplary embodiment of the invention presented, seal electrode 406B is horseshoe shaped and runs about the perimeter of jaw 402.

A raised cut electrode 408 runs along the center of jaw 402, between the arms of seal electrode 406B. The exposed top of cut electrode 408 conducts electrical energy from the electrical generator 102A,102B sufficient to cut tissue held between the jaws 402 and 404. Cut electrode 408 may be electrically isolated from seal electrode 406B by an insulation sleeve 409. In the embodiment illustrated, sleeve 409 surrounds the sides of cut electrode 408, leaving the top of cut electrode 408 exposed. Sleeve 409 may be formed of a high temperature resistant material with high dielectric property such as silicone or ceramic to avoid electrical shorts between a higher potential cut electrode 408 (˜300V-750V) and a lower potential coag/seal electrode 406A (˜50V-200V). If an electrical short were to form between these two poles, inadequate energy would be delivered to the tissue resulting in an incomplete seal and/or cut. Due to the high temperatures generated on the cut electrode 408 during activation, materials with lower heat resistance would degrade.

End effector 202 is coupled to clevis 209 at pivot axis A by pivot pin 411 and pivot fastener 414. In the exemplary embodiments, clevis 209 is a clam shell shaped structure with the jaws 402 and 404 coupled between the two halves of the clam shell. At the tip of jaw 404 may be found an end cap 407. End cap 407 may prevent arcing between cutting tip 408′ and seal electrode 406B.

Jaws 402 and 404 may comprise drive ends that are substantially flat. The drive ends 420 and 421 of jaws 402 and 404 may be found opposite the working portion of the jaws. The drive ends lie generally parallel to each other with sliding contact to cause a rotational engagement of the jaws. The jaws 402 and 404 are co-axially pivoted about a medial point by means of pivot pin 411. Cam channel 403A and 403B are formed in the drive ends 420 and 421 of respective jaws 402 and 404. The cam channels 403 and 403B are used to transfer mechanical motion of the rotatable drivers 234 to actuate jaws 402 and 404.

Cam channels 403A and 403B are disposed at a slant relative to a clevis channel 414 running along the length of clevis 409. Actuating pin 405, threads through clevis channel 414 and both cam channels 403A and 403B. Clevis channel 422 guides the movement of actuating pin 405 as it is moved back and forth relative to pivot pin 405. Sliding movement of actuating pin 405 along clevis channel 422 causes corresponding movement of cam channels 403A and 403B which rotates jaw 402 and 404 about pivot pin 411. As actuation pin 405 moves longitudinally back and forth within clevis channel 422, the drive ends 420 and 421 of the jaws are forced to move along their respective slanted channels 403A and 403B. The movement of the cam channels 403A and 403B causes a corresponding rotation of the end effector jaws 402 and 404 about the pivot axis A.

A detailed disclosure of the actuation of jaws 402 and 404 may be found in U.S. Pat. No. 9,055,961 issued Jun. 16, 2015 to Scott Manzo et al, particularly FIG. 3-5 and columns 12, line 57 to column 16, line 45; U.S. Patent Publication 2012/0310221 by Kevin Durant et al.; and U.S. Patent Publication 20120310254 by Scott Manzo et al., all of which are incorporated herein by reference.

Continuing now to FIG. 4D, a perspective view of the electrosurgical bipolar end effector with jaws 402 and 404 in an open position. Jaw 404 generally comprises a sealing electrode 406A, an elastomeric layer 419 below the seal electrode 406A and a rigid jaw base supporting the elastomeric layer 419.

### Compliant Electrode

In jawed end effectors, it is advantageous to maintain consistent pressure on tissue between the jaws. In order to maintain consistent pressure, surgical instruments with rigidly fixed electrodes required tight manufacturing tolerances that resulted in low process capability and high scrap or reject rate. Because of the tight tolerances, low process capability, and high scrap rate a fixed electrode jaw may have a higher cost of manufacture.

In embodiments of the invention, seal electrode 406A is made compliant by the elastomeric properties of elastomeric layer 419. Elastomeric layer 419 provides a compliant spring like surface below the seal electrode 406A allowing the seal electrode 406A to “float”. Typically, seal electrode 406A may be composed of a rigid metal, e.g. sheet metal. Slight movement of the seal electrode 406A helps maintain consistent pressure on tissue held by the jaws 402, 404. The elastomeric layer 419 allows the seal electrode 406A to be more compliant, and therefore more tolerant of jaw misalignment. In some cases, the elastomeric layer 419 may also be an insulator and serve to electrically isolate the seal electrode 406A from a conductive base or electrode within the jaw 404 that may be coupled to a different voltage or ground.

Elastomeric layer 419 may be made of a suitable material selected for the operating conditions of a surgical site. Due to the high heat produced by the cut electrode 408, elastomeric layer 419 is preferably a surgery suitable material; e.g. non-toxic, that is also heat resistant and has a high dielectric property. The preferred material for the elastomeric layer 419 is silicone.

In this illustration, jaw 404 is shown below jaw 402, and the seal electrode 406A of jaw 404 is prominently displayed. Electrical conductor 412 provides energy to seal electrode 406A. In some embodiments of the invention, seal electrode 406A is made of a metal formed into a horseshoe or U-shape. Seal electrode 406A is similar to seal electrode 406B in that it is substantially flat and horseshoe shaped. Openings in sealing electrode 406A allow an elastomeric layer 419 below the sealing electrode 406A to be overmolded to form spacers 416. Spacers 416 prevent electrodes 406A and 406B from contacting each other when jaws 402 and 404 are in a closed position to prevent or at least mitigate a short circuit. When jaws 402 and 404 are in a closed position, spacers 416 leave an airgap between seal electrode 406A and 406B.

Between the arms of seal electrode 406A is an elastomeric strip 418. Elastomeric strip 418 is positioned opposite cut electrode 408 when the jaws 402 and 404 are in a closed position. Elastomeric strip 418 is preferably heat resistant and non-conductive. The return path for both cut electrode 408 and seal electrode 406B is seal electrode 406A. The elastomeric strip 418 functions much like a spring to provide counter pressure to tissue pressed by the cut electrode 408. The compliance of the elastomeric strip 418 may serve to push tissue up into the cut electrode 408 (or other cutting device) during cutting as portions of the tissue desiccate (and shrink) from the heat of the cut electrode 408.

Referencing FIG. 4E, a perspective view of jaw 402 illustrated as isolated from the clevis 209 and wrist structures 203 to show the jaw 402 and in greater detail. Jaw 402 includes an opening 422 for receiving the pivot pin 411. Actuating pin 405 (not shown) slides along cam channel 403A causing a rotation of jaw 402 about pivot pin 411. Cam channel 403A is slightly slanted causing jaw 402 to rotate in an arc about a pivot axis through opening 422 as actuating pin 405 moves horizontally.

FIG. 4F illustrates a side cross sectional view of jaw 402. Cutting electrode 408 is mounted to jaw 402 at mounting tab 424 and 426. Electrical conductor 410 is coupled to cut electrode 408. In some embodiments of the invention, cut electrode 408 and cutting tip 408′ are unitary structures. As illustrated in FIG. 4F, cutting tip 408′ is an extension of cut electrode 408, extending out of the distal end of jaw 402. In embodiments where cut electrode 408 and cutting tip 408′ are unitary, electrical conductor 410 provides energy to both. As mentioned previously, cam channel 403A is slanted relative to a horizontal line through opening 422. An actuating pin traveling along said horizontal line would cause a slight rotation of cam channel 403A and a corresponding rotation of jaw 402 about pivot pin 411.

FIG. 4G illustrates a perspective view of jaw 404 isolated from the rest of the end effector 202. In between the two arms of seal electrode 406A is an elastomeric strip 418 of elastomeric material. This elastomeric strip 418 may be formed separate from elastomeric layer 419 or alternatively molded with elastomeric layer 419. Due to its elastic material properties, the elastomeric strip 418 places upward pressure on tissue as jaws 402 and 404 when the jaws close on the tissue. Elastomeric strip 418 acts much like a spring or trampoline pushing up on the tissue as the tissue is being cut. As cut electrode 408 cuts a vessel or tissue draped between the jaws 402 and 404, the elastomeric strip pushes up on the tissue; maintaining more consistent contact between the tissue and the cutting electrode 408 (of other cutting device). Inconsistent contact with the cut electrode 408 may result in an incomplete cut.

In FIG. 4H, jaw 404 is illustrated in a side view. Conductor 412 is coupled to jaw 404 and provides electrical energy to the seal electrode 406A. Spacers 416 rise slightly above the plane of the seal electrode 406A. Spacers 416 may be unitary with elastomeric layer 419 and protrude out of openings in seal electrode 406A. When jaws 402 and 404 are closed, spacer 416 prevents seal electrodes 406A and 406B from contacting. An airgap is thereby left between electrodes 406A and 406B when jaws 402 and 404 engage tissue. The airgap prevents or mitigates shorts between electrodes 406A and 406B should they touch.

FIGS. 4I and 4J, cross sectional side views of end effector 202, illustrate end effector 202 in an open and closed configuration respectively. With reference to FIGS. 4I-4J, each jaw 402, 404 has a corresponding generally slanted slot 403A, 403B. Slots 403A, 403B are opposite and distal from the jaw portion of the end effectors 402 and 404. Connector 430 couples to each jaw 402,404 by an actuation pin 405 which passes through both slots 403A, 403B and has an axis generally parallel and proximal to pivot pin 411. The slots 403A, 403B are angularly offset from pivot pin 411, so that the line extensions of the slots do not pass through pivot pin 411. The actuation pin slides along slots 403A,403B in a cam-slot engagement, causing the jaws to rotate about pivot pin 411. As connector 430 slide distally, the jaw open apart from one another, as shown by FIG. 4I. As connector 430 slide proximally, the jaw close upon one another in a shearing action as shown in FIG. 4J.

Although a combination electrosurgical tissue cutter and sealer with cautery tip is specifically described in FIG. 4A-7, it should be appreciated that other surgical tools may be substituted without deviating from the scope of the invention.

For example, a mechanical knife such as detailed in U.S. Pat. No. 9,055,961 issued Jun. 16, 2015 to Scott Manzo et al., incorporated herein by reference, may be combined with an elastomeric layer 419 and seal electrode 406A. In a mechanical knife embodiment, the cut electrode 408 would be replaced with a mechanical knife, e.g. the mechanical knife disclosed in U.S. Pat. No. 9,055,961. The compliant seal electrode 406A may increase cautery efficiency due to more consistent contact between the seal electrode 406A and the tissue. The elastomeric layer 419 also allows the seal electrode 406A to be more compliant, and therefore more tolerant of jaw misalignment leading to lower manufacturing cost from discards.

A bipolar grasper with compliant electrode would also be within the scope of the invention. The compliant seal electrode 406A would allow the bipolar grasper to better cauterize vessels as elastomeric layer 419 would form a compliant spring like surface below the seal electrode 406A allowing the seal electrode 406A to “float”. The compliant seal electrode 406A would help maintain consistent pressure on tissue held by the jaws 402, 404 allowing for a better grip on the tissue.

Other types of surgical tools may benefit from an elastomeric layer 419 and compliant electrode 406A. For example, adaptations of bipolar forceps, fenestrated bipolar forceps, bipolar dissector etc. with compliant electrodes would be within the abilities of a person of ordinary skill and within the scope of the invention.

### Metalized Elastomeric Electrode

Previous embodiments disclosed the seal electrodes 406A-406B as metallic electrodes, e.g. sheet metal electrodes. The sealing electrode 406A is disposed over an elastomeric layer 419. Due to the elastic nature of the elastomeric layer 419, the sealing electrode 406A “floated” and was thereby more compliant and more tolerant of jaw misalignment than a rigidly fixed electrode.

Referring now to FIG. 4K, a compliant elastomeric seal electrode 406A′ is disclosed in place of the sealing electrode 406A and the underlying elastomeric layer 419. The compliant elastomeric seal electrode 406A′ may be formed by adding a conductive material into the elastomeric material. The resulting conductive elastomeric compound may then be molded or otherwise formed into the shape of the elastomeric seal electrode 406A′ in jaw 404.

The elastomeric seal electrode 406A′ is substantially flat and horseshoe shaped. The elastomeric seal electrode 406A′ on jaw 404 is placed opposite seal electrode 406B on jaw 402. In a close position of jaws 402 and 404, the elastomeric seal electrode 406A′ provides a slightly flexible surface to grip tissue and seal tissue.

An elastomeric material such as silicone may be impregnated with a conductive material such as carbon or stainless steel. Preferably, the conductive material should be able to form conductive pathways through the normally insulative silicone without sacrificing too much flexibility. Carbon fibers are generally small and thin enough, that if used as the conductive material to impregnate silicone, will still maintain the elastomeric property of silicone.

As noted previously, the elastomeric seal electrode 406A′ is preferably compliant to account for minor jaw misalignment and manufacturing tolerance. As with seal electrode 406A, elastomeric seal electrode 406A′ would be more compliant and thus more tolerant of jaw misalignment than a rigidly fixed electrode. The elastic property of the elastomeric seal electrode 406A′ would also allow the jaws 402 and 404 to maintain more consistent pressure across tissue grasped by the jaws. Difference in pressure due to jaw misalignment or position of the tissue with respect to the jaws may be ameliorated somewhat by the flexibility or “give” in the elastomeric seal electrode 406A′.

Spacers 416 were previously disclosed as “bumpers” to prevent the jaws 402 and 404 from physically contacting when the jaws close. In the embodiment of the invention, illustrated in FIG. 4K, the spacers 416 serve the same function. It should be noted that the spacers 416 are non-conductive. In other words, unlike the elastomeric seal electrode 406A′, the spacers 416 are not impregnated with a conductive material.

In the illustrated embodiment, the elastomeric seal electrode 406A′ is only shown on jaw 404. However, it should be evident to a person of ordinary skill that a second elastomeric seal electrode manufactured out of the same metalized elastomeric compound as that of elastomeric seal electrode 406A′ may be formed in the shape of the seal electrode 406B to replace seal electrode 406B in the jaw 402 as shown in FIG. 4G.

Forming a conductive elastomeric seal electrode 406A′ may have some advantages as compared to using a separate elastomeric layer 419 and metallic seal electrode 406A discussed previously. For example, a unitary conductive elastomeric seal electrode 406A′ may simplify the manufacturing process by reducing the number of parts to be assembled. Fewer parts in a surgical tool may also be easier to sterilize.

As with the separate elastomeric layer 419 and metallic seal electrode 406A discussed previously, the inventive concept of a conductive elastomeric seal electrode 406A′ may be beneficial to many surgical tools. For example, a mechanical knife such as detailed in U.S. Pat. No. 9,055,961 issued Jun. 16, 2015 to Scott Manzo et al., may be combined with conductive elastomeric seal electrode 406A′. The conductive elastomeric seal electrode 406A′ may increase cautery efficiency due to more consistent contact between the seal electrode 406A and the tissue. The conductive elastomeric seal electrode 406A′ would be more compliant than a fixed seal electrode, and therefore more tolerant of jaw misalignment leading to lower manufacturing cost from discards.

A bipolar grasper with compliant electrode would also be within the scope of the invention. The compliant conductive elastomeric seal electrode 406A′ would allow the bipolar grasper to better cauterize vessels. The conductive elastomeric seal electrode 406A′ may also help maintain consistent pressure on tissue held by the jaws 402, 404 allowing for a better grip on the tissue.

Other types of surgical tools may benefit from conductive elastomeric seal electrode 406A′. For example, adaptations of bipolar forceps, fenestrated bipolar forceps, bipolar dissector etc. with compliant electrodes would be within the abilities of a person of ordinary skill and within the scope of the invention

### Spring Functionality of Elastomeric Strip

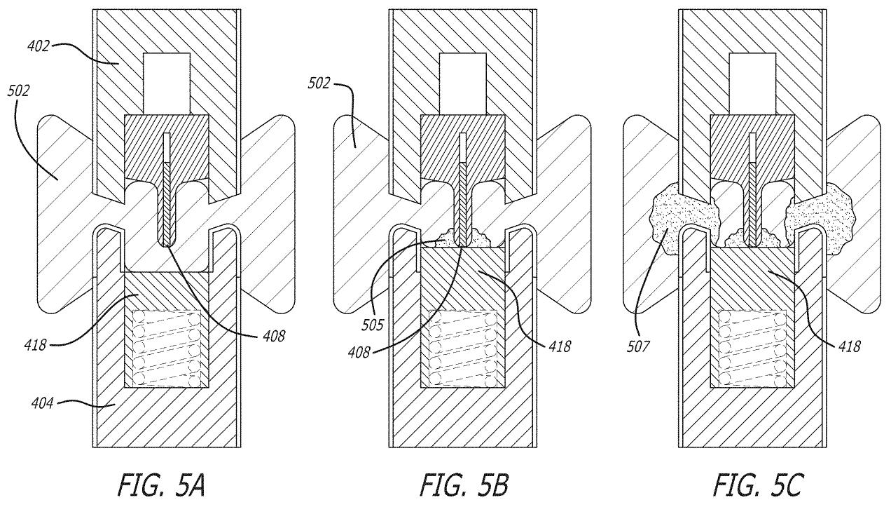

FIG. 5A-5C are front views of a functional representation of an exemplary electrical end effector 202 as it grasps, cuts and seals tissue. The elastomeric strip 418 is functionally represented by spring, however it should be understood that the elastic properties of the elastomeric strip 418 functions as a spring and an actual spring is unnecessary in this embodiment of the invention.

In FIG. 5A, tissue 502 is clamped between jaws 402 and 404 before activation of the generator 102A. In FIG. 5A, the cut electrode 408 and seal electrode 406A are not yet activated or energized to be deemed “hot”. Tissue 502 is slightly compressed by mechanical action of jaws 402 and 404, but the tissue 502 is not yet being cut nor sealed by the electrodes. As jaw 402 presses down on the tissue, elastomeric strip 418 presses up against the tissue 502 in an opposite elastic reaction similar to a trampoline.

In FIG. 5B, the generator 102A is activated and electrical energy is applied to the end effector 202. As tissue 502 desiccates under cut electrode 408 a void pocket 505 may form in the tissue. The void pocket 505 would normally cause the cut electrode to lose contact with the tissue and causing an incomplete or wispy cut. However, due the elastomeric strip 418 pressing up on the tissue 502, contact between tissue 502 and cut electrode 408 is maintained, and a complete cut can be made.

FIG. 5C illustrates the seal electrodes 406A-406B desiccating the tissue 502 at the perimeter of the jaws 402,404. The seal electrodes 406A-406B form pockets of dried tissue 507 that seals the cut ends of the tissue 502 that was cut by the cut electrode 408. As illustrated in FIG. 5C, a slight air gap is maintained between the two seal electrodes 406A-406B to prevent short circuiting them together.

### Concurrent Sealing and Cutting

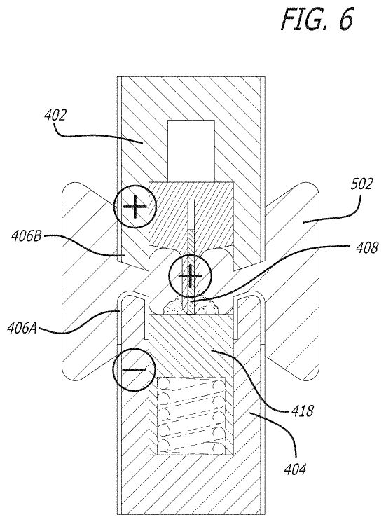

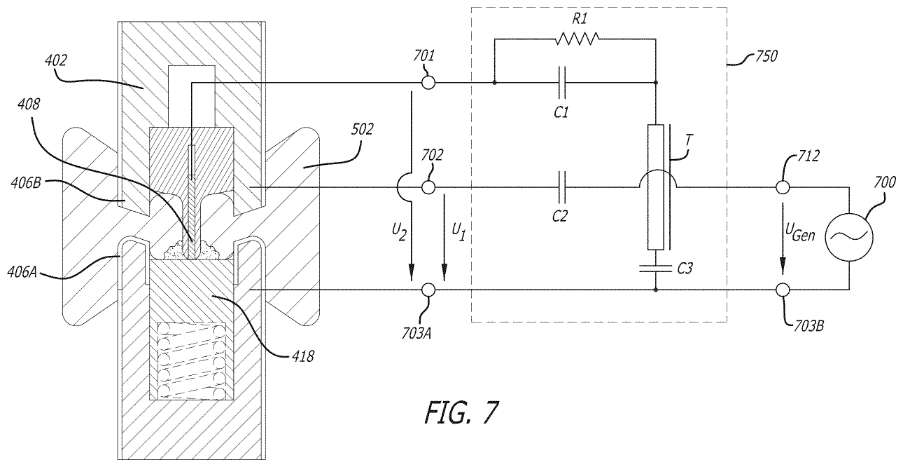

Referring now to FIGS. 6 and 7, a diagram is shown of the sealing and cutting electrodes to further discuss concurrent sealing and cutting of tissue 502 between the jaws 402,404. A generator, such as generator 102A shown in FIG. 1A, provides electrical energy to both the seal electrode 406B and the cut electrode 408 when energy is activated. The voltage applied to the cut electrode 408 is greater than the voltage applied to the seal electrode 406B. To seal tissue, a first voltage U₁ is applied between the sealing electrode 406B and the sealing or common electrode 406A. Concurrently to cut tissue, a second voltage U₂ greater than the first voltage U₁ is concurrently applied between the cutting electrode 408 and the common electrode 406A.

In accordance with one embodiment, the generator generates the first voltage U₁, an alternating current (AC) voltage, with a magnitude within the range of 50-200 volts (inclusive) for coagulation. The second voltage U₂ is stepped up or generated with a magnitude in the range of 300-750 volts (inclusive) for cutting. The second voltage U₂, an AC voltage, may be out of phase with the first voltage U₁.

In FIG. 7, a schematic diagram of the electrical circuit formed between tissue and an AC voltage generator 700 is shown. The generator 700 generates a generator voltage U_{Gen} that is coupled into the input nodes of an adapter circuit network 750. The adapter circuit network 750 is coupled between the generator 700 and the electrodes of the jaws of the remote controlled electrosurgical tool. Three electrical nodes (or poles) 701-703 are shown for applying different voltages to the sealing and cut electrodes of the jaws of the remote controlled electrosurgical tool. Node 701 is coupled to the cut electrode 408. Node 702 is coupled to seal electrode 406B. Node 703A is coupled to the electrode 406A that is common to both so it may also be referred to as a common electrode. Terminal 712 and 703B are terminals of the network 750 that couple to generator 700. Node 703A and terminal 703B are coupled together in a feed through path.

The adapter circuit network 750 filters the power provided by the generator 700. The adapter circuit network 750 includes capacitor C2 to filter out lower frequencies in the voltage signal U_{Gen} to form the sealing voltage U₁. Capacitor C3 of the adapter circuit network 750 similarly filters out lower frequencies in the voltage signal U_{Gen} before the transformer T.

The adapter circuit network further steps up the amplitude of the sealing voltage U₁ provided by the generator to form the cutting voltage U₂. The transformer T (a form of an inductor) in series with the parallel resistor R₁ and capacitor C₁, transforms (steps up or multiplies) the AC voltage from the generator into the higher cut voltage U₂. The transformer T applies a ratio of cut voltage to generation voltage of about 4.5 to 1. Accordingly, if the sealing voltage U₁ is 150V in magnitude from the generator, it is stepped up 4.5 times or up to 675V in magnitude as the cutting voltage U₂. Although a ratio of 4.5 to 1 has been proven to be effective, other ratios may also be used without deviating from the scope of the invention.

The adapter circuit network 750 may be included as part of the electrosurgical surgical instrument 101A. Alternatively, the adapter circuit network 750 may be part of the electrosurgical cabling between the control cart and the instrument 101A. In an alternate embodiment, the voltage step-up may be handled internally by the generator 700 with three wire cables routed between the generator and the electrosurgical instrument 101A.

The simultaneous application of both sealing and cutting voltages to concurrently cut and seal tissue may be advantageous over a serial application, e.g., sealing the vessel first then cutting it. When a vessel is sealed by electrical energy it is oftentimes desiccated by the intense heat. Cutting through desiccated tissue may be more difficult and as a result an incomplete cut may occur. Accordingly, it may be more advantageous to simultaneously apply energy to the seal and cut electrodes so that the tissue is cut just before or contemporaneous to sealing the cut tissue ends.

### CONCLUSION

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. This disclosure contemplates other embodiments or purposes.

For example, it will be appreciated that one of ordinary skill in the art will be able to employ a number of corresponding alternative and equivalent structural details, such as equivalent ways of fastening, mounting, coupling, or engaging tool components, equivalent mechanisms for producing particular actuation motions, and equivalent mechanisms for delivering electrical energy. Therefore, the above description should not be taken as limiting the scope of the invention which is defined by the appended claims.

1. An electrosurgical end effector comprising:
a first end effector jaw including a first seal electrode;
a second end effector jaw including a second seal electrode, wherein the second seal electrode is a compliant seal electrode;
a pivot pin extending through the first end effector jaw and the second end effector jaw, the pivot pin being configured to rotatingly couple the first end effector jaw and the second end effector jaw;
an actuation mechanism coupled to an end of at least the first end effector jaw to rotate at least the first end effector jaw about the pivot pin; and
a first electrical conductor to electrically couple at least one of the first seal electrode and the second seal electrode to a generator.

2. The electrosurgical end effector of claim 1, wherein the compliant seal electrode comprises elastomeric material impregnated with a conductive material.

3. The electrosurgical end effector of claim 1, wherein the compliant seal electrode comprises a sheet metal electrode disposed on top of an elastomeric material.

4. The electrosurgical end effector of claim 1, wherein the first end effector jaw further comprises a cutting device.

5. The electrosurgical end effector of claim 4, wherein the cutting device is a cut electrode.

6. The electrosurgical end effector of claim 4, wherein the cutting device is a mechanical knife.

7. The electrosurgical end effector of claim 1, further comprising:
a cutting tip at a distal end of the first jaw.

8. The electrosurgical end effector of claim 7, wherein the cutting tip is unitary with the cut electrode.

9. The electrosurgical end effector of claim 4, further comprising:
an elastomeric strip on the second jaw, opposite the position of the cutting device on the first jaw, to push tissue into contact with the cutting device.

10. The electrosurgical end effector of claim 1 further comprising:
elastomeric spacers on at least one jaw of the first and second jaws, the spacers to maintain an air gap between the first and second jaws when the first and second jaws are in a closed position.

11. An electrosurgical tool for a teleoperated surgical system, the electrosurgical tool comprising:
a pair of end effector jaws rotatingly coupled together at a pivot axis, a first end effector jaw of the pair of end effector jaws to pivot about the pivot axis, open and closed, with respect to a second end effector jaw of the pair of end effector jaws;
a first seal electrode coupled to the first end effector jaw and a second seal electrode coupled to the second end effector jaw, wherein the second seal electrode is a compliant seal electrode;
an actuation mechanism to open and close the pair of end effector jaws;
a shaft having a distal end to extend the pair of end effector jaws into a surgical site; and
an interface base coupled to a proximal end of the shaft, the interface base to couple to a robotic slave, the interface base including a first spool to control at least the first end effector jaw.

12. The electrosurgical tool of claim 11, wherein the compliant seal electrode comprises elastomeric material impregnated with a conductive material.

13. The electrosurgical tool of claim 11, wherein the compliant seal electrode comprises a sheet metal electrode disposed on top of an elastomeric material.

14. The electrosurgical tool of claim 11, wherein the first end effector jaw further comprises a cutting device.

15. The electrosurgical tool of claim 14, wherein the cutting device is a cut electrode.

16. The electrosurgical end effector of claim 14, wherein the cutting device is a mechanical knife.

17. The electrosurgical tool of claim 13, further comprising:
a cutting tip at a distal end of the first jaw.

18. The electrosurgical tool of claim 17, wherein the cutting tip is a portion of the cut electrode extending out of the distal tip of the first end effector jaw.

19. The electrosurgical tool of claim 14, further comprising:
an elastomeric strip on the second jaw, opposite the position of the cutting device on the first jaw, to push tissue into contact with the cutting device.

20. The electrosurgical tool of claim 11, further comprising:
a transformer to step up a first voltage to a second voltage, wherein the first voltage is sufficient to seal tissue and the second voltage is sufficient to cut tissue.

21-31. (canceled)
